(19) United States
(12) Patent Application Publication
(10) Pub. No.: US 2006/0127480 A1
(43) Pub. Date: Jun. 15, 2006

(54) Pharmaceutical excipients comprising inorganic particles in association with an organic polymeric material and forming a solid reticulated matrix, compositions, manufacturing and use thereof

(76) Inventors: Michael Tobyn, Wiltshire (GB); John Nicholas Staniforth, Avon (GB); Cheryl Clinch, Bath (GB); Matthew Paul Hearn, Thursby (GB); Dominic Walsh, Avon (GB); Simon Robert Hall, Devon (GB)

Correspondence Address: DAVIDSON, DAVIDSON & KAPPEL, LLC, 485 SEVENTH AVENUE, 14TH FLOOR, NEW YORK, NY, 10018, US

(21) Appl. No.: 10/531,073

(22) PCT Filed: Oct. 10, 2003
(86) PCT No.: PCT/GB03/04463

§ 371 Date: Dec. 16, 2005

(30) Foreign Application Priority Data
Oct. 11, 2002 (US) 60418055

(51) Int. Cl. A61K 9/14

(52) U.S. Cl. 424/484; 977/906

(57) ABSTRACT

A pharmaceutical excipient comprising a solid, reticulated matrix, wherein the matrix comprises an aggregation of inorganic particles in association with an organic polymeric material, defines a plurality of pores with a mean width in the range of about 0.01-500 m, and has a specific surface area of at least about 1 m2/g is described, as are products comprising such excipients, methods of making them and use thereof.

The present invention telates in part to a pharmaceutical excipient, and to its preparation and use in pharmaceutical products, especially oral solid dosage forms. The present invention further relates in part to the use of the pharmaceutical excipient of the invention to increase the oral bioavailability of poorly water soluble drugs.

The present invention further relates to pharmaceutical products, to processes of preparing the same and to uses thereof. In particular, the present invention relates to pharmaceutical products comprising one or more therapeutic agents having poor solubility in the physiological fluids present in the gastrointestinal tract of a patient. Pharmaceutical products for oral administration to an animal patient, in particular a human patient, may be presented in a variety of oral dosage forms, including tablets, capsules, powders, granules, pellets or the like. Tablets may be made by compression, moulding or granulation of a therapeutic agent, optionally together with one or more accessory pharmaceutically acceptable ingredients. Compressed tablets may be prepared by compressing in a suitable machine a therapeutic agent in a free flowing from, such as a powder or granules, optionally mixed with a binder, lubricant, inert diluent, dispersing agent or the like. Moulded tablets may generally be made by moulding in a suitable machine a mixture of a therapeutic agent in powdered form moistened with an inert liquid diluent. The tablets may optionally be coated. Capsules, which may be of the hard or soft type, generally comprise an outer shell which may be composed of, for example hydroxpropylmethylcellulose or gelatin, and an inner core comprising a therapeutic agent which can typically be provided in granular, powder or liquid form.

Delivery by oral administration can be particularly desirable for many therapeutic agents. Furthermore, oral administration can be desirable due to the non-invasive nature thereof and also the substantially accurate dosing control that can generally be achieved with oral administration. Oral administration can also be advantageous in terms of patient acceptability and, therefore, improved patient compliance.

A problem that can be encountered with oral administration, however, is where the therapeutic agent to be administered exhibits poor solubility in the physiological fluids present in the gastrointestinal tract of a patient. Poorly water soluble drugs present significant challenges during formulation of, e.g., an oral dosage form. A high proportion of new chemical entities are characterized by an unfavorable solubility profile. In such cases, complete or even substantial dissolution may not occur during the passage of the therapeutic agent through the gastrointestinal tract (a time period of the order of up to 48 hours). Furthermore, such dissolution may be variable from one administration to the next and may also be patient dependent. Consequently, the therapeutic agent may not be fully available, or substantially not reproducibly available, for absorption into the general circulation of the patient. The above can be problematic in terms of wastage of the therapeutic agent, but more importantly, in terms of achieving accurate dosing and substantially consistent bio-availability thereof. Furthermore, these problems have recently been exacerbated by the increase in production of poorly soluble compounds by drug discovery methods, such as combinatorial chemistry, and also a general trend in dosage decrease for therapeutic agents. The formulation challenges due to poor water solubility include attempts to avoid the undesirable product characteristics that typically result, including but not limited to slow onset of action, low oral bioavailability and variability of drug absorption related to the presence or absence of food.

The problem of improving the bio-availability of such poorly and variably soluble therapeutic agents has been discussed in WO 00/09093. WO 00/09093 describes pharmaceutical compositions adsorbed onto solid particles which may be further formulated into solid dosage forms. The compositions and dosage forms taught by WO 00/09093 are described as improving the bio-availability of a wide range of therapeutic agents, including therapeutic agents that are known to have or suspected of having poor bio-availability. WO 00/09093 also discusses how powdered solution technology had previously been proposed as a technique for the delivery of water-insoluble therapeutic agents, Spireas et al, “Powdered Solution Technology: Principles and Mechanisms, Pharm. Research, Vol. 9, No. 10 (1992) and Sheth, A. and Jarowski, C. I., “Use of powder solutions to improve the dissolution rate of polythiazide tablets,” Drug Development and Industrial Pharmacy, 16 (5), 769-777 (1990). The concept of powdered solutions involved converting solutions of therapeutic agents or liquid therapeutic agents into a dry, nonadherent, free-flowing compressible powder by admixing the liquid therapeutic agents or solutions of therapeutic agents with a selected carrier. Although the therapeutic agent was in a solid form, it was held in a solubilised liquid state, which increased the wetting properties of the therapeutic agent, and therefore enhanced the dissolution. However, the application of powder solution technology was limited because the resulting admixture powder generally had poor and erratic flowability and compressibility properties.

The present invention, however, now alleviates the above described problems hitherto associated with poorly soluble therapeutic agents, in terms of increasing the bio-availability, and also the reproducibility of such bioavailability, of such therapeutic agents whilst also providing a pharmaceutical product exhibiting good flow and compressibility characteristics which were not hitherto achieved with the above described powder solution technology. Furthermore, pharmaceutical products as provided by the present invention can be advantageous in allowing the therapeutic agent or agents to remain substantially wholly in a solid state until a time following administration, thereby substantially obviating chemical instability often associated with liquid state chemicals. Pharmaceutical products as provided by the present invention can, therefore, be particularly suitable for oral administration due to the desirable dissolution rate in the physiological fluids of the gastrointestinal tract that can be achieved for therapeutic agents as provided by pharmaceutical products according to the present invention.

It is an object of the invention to provide a pharmaceutical excipient for use with poorly water-soluble therapeutically active agents.

It is a further object of the invention to provide a pharmaceutical product comprising a therapeutically active agent together with a pharmaceutical excipient of the present invention.

It is a further object of the invention to provide a pharmaceutical excipient which has a high surface area and into which a poorly soluble drug can be incorporated to enhance the dissolution profile with the final objective of enhancing the oral bioavailability of said drug.

The present invention is further directed to a pharmaceutical product comprising one or more active agents intimately associated with (e.g, coated onto the surface of) the pharmaceutical excipient of the present invention.

In certain preferred embodiments, the active agent (e.g., drug) is combined with the pharmaceutical excipient of the present invention in an amount from about 1 to about 50% by weight.

In certain preferred embodiments of the present invention, the pharmaceutical excipient comprises from about 5 to about 95% by weight organic polymer, preferably as a template, with the remainder comprising inorganic material. In further preferred embodiments, the polymer comprises from about 20 to about 80% w/w of the pharmaceutical excipient.

The present invention is further directed to an oral solid dosage form comprising a unit dose of one or more active agents intimately associated with (e.g, coated onto the surface of) the pharmaceutical excipient of the present invention, the oral solid dosage form being, for example, a pharmaceutical powder, a capsule, or a tablet.

Typically, a pharmaceutical excipient product according to the present invention comprises a support material for the therapeutic agent, which support material can be an organic or inorganic support material having a reticulated microstructure substantially as hereinafter described in greater detail.

In accordance with the above objects and others and in a first aspect, the present invention provides a pharmaceutical excipient comprising a solid, reticulated matrix, wherein the matrix comprises an aggregation of inorganic particles in association with an organic polymeric material, defines a plurality of pores with a mean width in the range of about 0.01-500 μm, and has a specific surface area of at least about 1 m²/g. Preferably, the mean width of the pores is in the range of about 0.1-500 μm and it is preferred that the matrix has a specific surface area of no more than about 100 m²/g.

The pores in the matrix are not generally of a uniform shape or cross-section. For example, when many embodiments of the invention are viewed with a scanning electron microscope, although a few appear to be circular in cross-section, most of the pores appear to have irregular cross-sections. For the purpose of this specification, the mean width or size of the pores in a sample or portion of the matrix, or of any other pharmaceutical product described in this specification, is defined as being equal to the diameter of a circle that bounds an area equal to the mean of the pores' apparent cross-sectional areas. It can be determined as follows. A scanning electron micrograph of a sample or portion of the matrix or other product is taken at a magnification and from a direction which clearly shows a pore distribution that is representative of the sample or portion of matrix or other product. The apparent cross-sectional area of each of the visible pores is then measured and the mean of these measured values is calculated. The diameter of the circle that bounds an area equal to the mean of the pores' apparent cross-sectional areas is then calculated from this latter value. The apparent cross-sectional area of a pore is the area of the cross-section of that pore which is apparent or visible in the scanning electron micrograph. The “width” of an individual pore is related to its apparent cross-sectional area in a similar manner and can be determined in a similar way.

Likewise, the inorganic particles are not always of a uniform shape or cross-section. For example, when certain embodiments are viewed with a scanning electron microscope, most of the inorganic particles appear to have irregular cross-sections.

For the purpose of this specification, the mean width or size of the inorganic particles in a sample or portion of the matrix, or of any other pharmaceutical product described in this specification, is defined as being equal to the diameter of a circle that bounds an area equal to the mean of the inorganic particles' apparent cross-sectional areas. It can be determined as follows. A scanning electron micrograph of a sample or portion of the matrix or other product is taken at a magnification and from a direction which clearly shows an aggregation of inorganic particles that is typical of the sample or portion of matrix or other product. The apparent cross-sectional area of each of the visible inorganic particles is then measured and the mean of these measured values is calculated. The diameter of the circle that bounds an area equal to the mean of the inorganic particles' apparent cross-sectional areas is then calculated from this latter value. The apparent cross-sectional area of an inorganic particle is the area of the cross-section of that particle which is apparent or visible in the scanning electron micrograph. The “width” of an individual inorganic particle is related to its apparent cross-sectional area in a similar manner and can be determined in a similar way. When the inorganic particles are substantially spherical, it follows from the foregoing that their mean width is equal to their mean diameter. If, as can be the case in certain aspects and embodiments of the invention, the inorganic particles are fused together, their apparent cross-sectional areas can be measured with reference to the grain boundaries that will still be apparent in such a product.

The term “specific surface area” is used in this specification to denote the surface area per unit weight of a material, as determined according to International Standard ISO 9277 “Determination of the specific surface area of solids by gas adsorption using the BET method.” (reference number ISO9277:1995(E))

In preferred embodiments of the first aspect of the invention, the mean width of the pores is within a range of about 0.5-300, 1-200, 3-100, 5-80, 15-70, or 20-60 μm. In other embodiments, the mean width of the pores is within a range of about 0.1-10, preferably about 0.1-5 μm, and, more preferably about 0.3, 2 or 3 μm. In further preferred embodiments, the matrix has a specific surface area of at least about 2, 3, 4, 5, 10 or 20 m²/g and, preferably, of up to about 100, 50 or 40 m²/g.

It is preferred for the inorganic particles employed in the first aspect of the invention to be crystalline and for the aggregation of inorganic particles to comprise a plurality of discrete but possibly abutting crystals. The mean width of the inorganic particles or crystals so employed is preferably about 0.1-50 μm and more preferably less than about 30, 25, 15, 10, 5 or 1 μm and, even mote preferably, about 0.1-25, 0.2-10 or 0.2-5 μm. In some arrangements, the mean width of the particles or crystals of inorganic material can be between about 0.1 and about 0.2 μm, or between about 1 and about 15 μm.

In further preferred forms of the excipient in accordance with the first aspect of the invention, the pores comprise primary and secondary pores, wherein the primary pores have a mean width of about 2-500 μm and are defined between structural elements formed from the matrix, the secondary pores have a mean width of 0.01-10 μm and are defined within said structural elements, and the mean width of the secondary pores is less than the mean width of the primary pores.

In accordance with a second aspect of the invention there is provided a pharmaceutical excipient comprising a solid, reticulated matrix, wherein the matrix comprises an inorganic material in association with an organic polymeric material, a plurality of primary pores with a mean width of about 2-500 μm are defined between structural elements formed from the matrix, a plurality of secondary pores with a mean width of about 0.01-10 μm are defined within said structural elements, and the mean width of the secondary pores is less than the mean width of the primary pores.

The excipient, in preferred embodiments of the first aspect of the invention, can have a specific surface area of at least 0.1, 0.5, 1, 3, 4, 5, 10 m²/g and, more preferably, of up to 100, 50 or 40 m²/g.

In preferred embodiments of the first or second aspect of the invention, the mean width of the primary pores is at least about 5, 10, 20 or 40 μm and, preferably, no more than about 300, 200, 100 or 50 μm. It is preferred that the mean width of the secondary pores is at least about 0.01, 0.05, or 0.1 μm and, preferably, no more than about 5, 3, 2, 1.5 or 1 μm. In particular embodiments of the first or second aspect of the invention, the mean width of the primary pores is within a range of about 2-50, 10-200, 10-500, 10-100, or 5-20 μm and the mean width of the secondary pores is within a range of about 0.1-5, 0.1-1, or >1 μm. Preferably, at least about 50, 55, 70, 80, 90, or 95% of the primary pores have a width, preferably an apparent width, that is greater than the mean width of the secondary pores. It is also preferred for at least about 50, 55, 70, 80, 90, or 95% of the secondary pores to have a width, preferably an apparent width, that is less than the mean width of the primary pores.

The structural elements can be in the form of primary walls that define the primary pores, and can comprise a network of secondary walls that define the secondary pores. The primary walls, preferably, have a mean width of about 10-500, 10-200, 20-100 or 10-50 μm. The secondary walls, preferably, have a mean width of about 0.01-5 or 0.5-2 μm.

In embodiments of the first or second aspect of the invention, the matrix can be in the form of a plurality of agglomerations of organic polymeric material and inorganic particles or material, in which the secondary pores are formed. In such embodiments, it is preferred that the primary pores are located between adjacent such agglomerations. The agglomerations can form a continuous structure defining a plurality of primary pores.

The inorganic material employed in excipients in accordance with the second aspect of the invention is preferably particulate and, in preferred embodiments, is crystalline and can comprise a plurality of discrete but possibly abutting crystals. The mean width of the particles or crystals of inorganic material is preferably in the range of about 0.1-50 cm. The mean width of the particles or crystals of inorganic material is preferably less than about 30, 25, 15, 10, 5 or 1 μm and, even more preferably, about 0.1-25, 0.2-10 or 0.2-5 μm. In some arrangements, the mean width of the particles or crystals of inorganic material can be between about 0.1 and about 0.2 μm, or between about 1 and about 15 μm.

The organic polymeric material can serve, in either the first or second aspect of the invention, to bind the inorganic particles or material into the matrix and can form a template for the inorganic particles or material. When the organic polymeric material forms a template for the inorganic particles or material, it is preferred for particles of the latter to be coated onto the polymeric template. In such embodiments, the secondary pores can be defined between adjacent inorganic particles.

In preferred embodiments of the first and second aspects of the invention, the matrix consists essentially or solely of an aggregation of inorganic particles in association with an organic polymeric material. In further preferred embodiments, the excipient consists essentially or solely of the matrix.

In certain preferred embodiments of the invention in its first or second aspect, the pharmaceutical excipient comprises from about 5 to about 95% by weight of said polymeric material or template, and the remaining portion of said pharmaceutical excipient comprises said inorganic particles or material. In certain embodiments, said polymeric material or template comprises from about 20 to about 80% by weight of said pharmaceutical excipient, and the remaining portion of said pharmaceutical excipient comprises said inorganic particles or material. Both the primary and secondary pores can be substantially spherical, especially the secondary pores.

In both the first and second aspect of the invention, the excipient is preferably particulate and can comprise, or consist essentially of matrix particles with a mean width of up to about 1000, 500, 300 or 250 μm and preferably of at least about 10, 50 and 100 μm.

The organic polymeric material employed in excipients in accordance with either the first or second aspect of the invention is preferably pharmaceutically acceptable and is preferably at least readily soluble (as defined in Martindale 31^{st} edition 1996) in water at between 20 and 50° C. within 24 hours.

The organic polymeric material can be or include a cellulose ether, especially hydroxyalkylcelluloses and carboxyalkylcelluloses, most especially hydroxyethylcellulose, hydroxypropylcellulose, hydroxyptopylmethylcellulose, cellulose derivatives, e.g. ethylcellulose, hydroxypropylcellulose, hydroxypropylmethylcellulose, methylcellulose, sodium carboxymethylcellulose, and the like, and mixtures thereof. Alternatively, a variety of polymers such as poly(amino acids), poly(amino acid esters), poly(carboxylic acids), poly(hydroxycarboxylic acids), polyorthoesters, polyphosphazenes, polyalkylene glycols and related copolymers may be employed. For example, poly(amino acids) such as poly-L-aspartic acid, poly(lysine), and poly(glutamic acid) may be utilized. Related copolymers such as poly(lactic acid-co-lysine) (PLA/Lys) and a poly(ethylene :glycol)-poly(aspararic acid) block copolymer may also be employed. Other polymers which are suitable for use in the present invention include copolymers of N-(2-hydroxypropyl)-methacrylamide (HPMA copolymers).

The polymeric material is preferably a pharmaceutically acceptable gum, including but not limited to xanthan gum, locust bean gum, galactans, mannans, vegetable gums such as alginates, gum karaya, pectin, agar, tragacanth, accacia, carrageenan, tragacanth, chitosan, alginic acid, other polysaccharide gums (e.g. hydrocolloids), and mixtures of any of the foregoing. Further examples of specific gums which may be useful in the present invention include but are not limited to acacia catechu, salai guggal, indian bodellum, copaiba gum, asafetida, cambi gum, Enterolobium cyclocarpum, mastic gum, benzoin gum, sandarac, gambier gum, butea frondosa (Flame of Forest Gum), myrrh, konjak mannan, guar gum, welan gum, gellan gum, tara gum, locust bean gum, catageenan gum, glucomannan, galactan gum, sodium alginate, tragacanth, chitosan, xanthan gum, deacetylated xanthan gum, pectin, sodium polypectate, gluten, karaya gum, tamarind gum, ghatti gum, Accaroid/Yacca/Red gum, dammar gum, juniper gum, ester gum, ipil-ipil seed gum, gum talha (acacia seyal), and cultured plant cell gums including those of the plants of the genera: acacia, actinidia, aptenia, carbobrotus, chickorium, cucumis, glycine, hibiscus, hordeum, letuca, lycopersicon, malus, medicago, mesembryanthemum, oryza, panicum, phalaris, phleum, poliathus, polycarbophil, sida, solanum, trifolium, trigonella, Afzelia africana seed gum, Treculia africana gum, detarium gum, cassia gum, carob gum, Prosopis africana gum, Colocassia esulenta gum, Hakea gibbosa gum, khaya gum, scleroglucan, zea, mixtutes of any of the foregoing, and the like.

A plurality of polymers can be included in the polymeric material and the most preferred polymeric materials are polysaccharides, proteins and mixtures thereof. Preferred examples include xanthan gum, dextran, acacia gum and egg albumen.

The inorganic material forming the inorganic particles employed in excipients in accordance with the first aspect of the invention, and the inorganic material used in excipients in accordance with the invention's second aspect, can be silica or, more preferably, a pharmaceutical acceptable alkaline earth metal salt, of which preferred examples include calcium phosphate, calcium carbonate, calcium sulphate, calcium silicate, magnesium hydroxide and calcium magnesium carbonate (dolomite). It will of course be appreciated that any polymorph of such alkaline earth metal salts may be employed and that selected such polymorphs may be particularly advantageous for use in the present invention. Preferably, the pharmaceutically acceptable inorganic material dissolves readily at a pH encountered along the gastrointestinal tract of a human being (e.g., at a pH of between about 1.6 and about 7.2). The most preferred alkaline earth metal salts are calcium phosphate and calcium carbonate. The most preferred forms of calcium phosphate are hydroxyapatite, brushite and tricalcium phosphate, and the most preferred forms of calcium carbonate are calcite and vaterite. In certain preferred embodiments, a plurality of inorganic materials are included in the excipient.

In certain embodiments of the present invention, the excipient or matrix includes an effervescent material. In certain embodiments the inclusion of the effervescent material will create bubbles within the matrix that can provide internal closed pores within the matrix. Certain effervescent materials for use in accordance with the present invention, include for example effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the acid component of the effervescent couple may be present.

In certain embodiments of the present invention, an excipient in accordance with the first or second aspect of the invention can be used as a precursor to an excipient in accordance with the third aspect of the invention (which is described below).

In a third aspect, the invention provides a pharmaceutical excipient comprising a porous network of fused inorganic elements, said network defining a plurality of pores with a mean width within the range of about 0.01-100 μm. Preferably, the fused elements have a mean width which is no more than about 10, 5 or 2 μm. In preferred embodiments, the inorganic elements have been fused by the action of heat on a plurality of discrete adjacent such elements.

It is in preferred for the inorganic elements to be at least partially crystalline.

In preferred embodiments of the excipient in accordance with the third aspect of the invention, the pores comprise primary and secondary pores, wherein the primary pores have a mean width of about 2-500 μm and are defined between structural elements formed from the matrix, the secondary pores have a mean width of 0.01-10 μm and are defined within said structural elements, and the mean width of the secondary pores is less than the mean width of the primary pores. Preferably, the mean width of the primary pores is at least about 5, 10, 20 or 40 μm and, preferably, no more than about 300, 200, 100 or 50 μm. It is preferred that the mean width of the secondary pores is at least about 0.01, 0.05, or 0.1 μm and, preferably, no more than about 5, 3, 2, 1.5 or 1 μm. Preferably, at least about 50, 55, 70, 80, 90, or 95% of the primary pores have a width that is greater than the mean width of the secondary pores. It is also preferred for at least about 50, 55, 70, 80, 90, or 95% of the secondary pores to have a width that is less than the mean width of the primary pores.

The structural elements can be in the form of primary walls that define the primary pores, and can comprise a network of secondary walls that define the secondary pores. The primary walls, preferably, have a mean width of about 10-500, 10-200, 20-100 or 10-50 μm. The secondary walls, preferably, have a mean width of about 0.01-5 or 0.5-2 μm.

In alternative embodiments, the excipient in accordance with the third aspect of the invention defines a plurality of pores with a mean width of 0.01-50 μm.

The inorganic elements are formed, preferably, from an inorganic material of the nature described above in the context of the first and second aspects of the invention.

Excipients in accordance with the third aspect of the invention, preferably, are particulate and can comprise, or consist essentially of particles with a mean width of up to about 500, 300 or 250 μm and preferably of at least about 10, 50 and 100 μm.

Excipients in accordance with the third aspect of the invention, preferably, consist essentially or solely of said fused inorganic elements.

Excipients in accordance with any of the aforementioned aspects of the invention can have a bulk density in the range of 0.25-1.5 g/cm³, mote preferably in the range of 0.25-0.75 g/cm³, and/or a tap density in the range of 0.5-2 g/cm³, more preferably in the range of 0.5-1 g/cm³.

Bulk density and tap density of the excipient may be measured using the following method: 10 ml of excipient are weighed into a 10 ml graduated measuring cylinder, avoiding agitation of the sample, and the mass is recorded. The sample is then tapped 50 times and the volume recorded. The tapping process is repeated until no change in volume is observed. Bulk density and tap density are calculated as the mass of excipient divided by the bulk volume or the tap volume respectively.

The present invention is also directed in part to a pharmaceutical excipient comprising a pharmaceutically acceptable alkaline earth metal salt in crystalline form coated onto the surface of a polymeric template comprising a pharmaceutically acceptable polymeric substance, said excipient having a specific surface area greater than 10 m²/g.

The invention is further directed in part to a pharmaceutical excipient comprising a porous matrix consisting essentially of a pharmaceutically acceptable alkaline earth metal salt in crystalline form in intimate association with a polymeric template, said excipient having a specific surface area greater than 10 m²/g.

Excipients in accordance with the present invention, are particularly advantageous in exhibiting high specific surface areas. Such high specific surface area excipients are particularly desirable for use with therapeutic agents exhibiting poor solubility in the physiological fluids of the gastrointestinal tract of a patient, in that they can aid in the dissolution of such agents in such an environment. In particular, by using it with an-excipient in accordance with the present invention, the dissolution rate (and advantageously the reproducibility thereof) of a poorly soluble therapeutic agent can be enhanced, in comparison to that achieved with a corresponding mass of the therapeutic agent with a conventional un-reticulated excipient.

The term “unit dose” as used herein denotes the amount of a therapeutic agent suitable for single administration and containing an effective amount of the agent to produce a desired therapeutic effect. The present invention achieves administration of such a unit dose of a therapeutic agent having poor aqueous solubility substantially as hereinafter described in greater detail to a patient during passage of the agent through the gastrointestinal tract of the patient. The term “administration” as used herein denotes administration of a therapeutic agent into the blood stream of a patient for systemic treatment, or into solution within a patient's gastrointestinal tract. The term “treatment” as used herein can include prophylaxis, as well as treatment of established conditions.

The terms “reticulated three-dimensional microstructure”; “reticulated microstructure”; “support”; “support material”; “scaffold”; and “construct” are considered for the purposes of the present invention to be alternative terms to describe the physical structure of the excipient product of the invention, comprising a matrix as herein before described. Thus, where these terms are used in this specification, the reader should understand that reference is being made to an embodiment of the excipient in accordance with the invention.

The term “template” as used herein is considered for the purposes of the present invention to describe a polymer structure (e.g., dextran, xanthan gum) onto or into which the inorganic material is crystallized or deposited. Thus, the combined materials (inorganic material and polymeric template) can form a construct, or an embodiment of the inventive excipient.

The term “therapeutically active agent” as used herein is considered for purposes of the present invention to be a chemical or biological agent which exhibits an effect when administered as a unit dose to a human patient.

Excipients and pharmaceutical products (reticulated three-dimensional microstructure or support) in accordance with any aspect of the present invention can have a specific surface area of at least 1 m²/g, preferably at least 2 m²/g and especially at least 5 m²/g. In principle, the specific surface area of a reticulated microstructure or support may be as high as is in practice achievable for the crystals thereof. Specific surface areas of up to 200 m²/g can be achieved for a reticulated three-dimensional microstructure or support employed according to the present invention. Typically a reticulated three-dimensional microstructure or support (excipient or pharmaceutical product) employed according to the present invention can have a specific surface area of up to 100 m²/g, or up to 50 m²/g. Preferred specific surface areas may be in the range of from 5 to 50 m²/g, more preferably from 10 to 40 m²/g.

A reticulated three-dimensional microstructure substantially as defined above more typically has a specific surface area of at least 2 m²/g and at least 5 m²/g. Again substantially as hereinbefore described it is preferred that such reticulated three-dimensional microstructure has a specific surface area of up to 100 m²/g, or up to 50 m²/g. Preferred ranges of specific surface areas are 5 to 50 m²/g and more preferably 10 to 40 m²/g.

The invention is further directed in part to a pharmaceutical excipient comprising a pharmaceutically acceptable alkaline earth metal salt in crystalline form coated onto the surface of a polymeric template comprising a pharmaceutically acceptable polymeric substance, said excipient having a bulk density in the range of 0.25-1.5 g/cm³, more preferably in the range of 0.25-0.75 g/cm³.

The invention is further directed in part to a pharmaceutical excipient comprising a porous matrix consisting essentially of a pharmaceutically acceptable alkaline earth metal salt in crystalline form in intimate association with a polymeric template, said excipient having a tap density in the range of 0.5-2 g/cm³, more preferably in the range of 0.5-1 g/cm³.

The invention is further directed in part to a pharmaceutical excipient comprising particles comprising a porous matrix of a polymer template and an alkaline earth metal salt in crystalline form defining a construct, said construct comprising strands defining primary walls having a mean width about 50 to about 500 μm, said primary walls comprised of (i) said polymer forming said polymeric template; (ii) aggregated crystals of said alkaline earth metal salt; and/or (iii) aggregated crystals of said alkaline earth metal salt coated on the surface of said polymer; said strands being arranged such that primary pores having a mean width from about 5 to about 300 μm are defined between at least two of said strands; said construct further comprising secondary-walls extending from surfaces of said strands, said secondary walls having a mean width from about 0.01 to about 5 μm and comprising crystals of said alkaline earth metal salt; said secondary walls being arranged such that said construct includes secondary pores having a mean width from about 0.01 to about 5 μm defined between at least two secondary walls.

In certain embodiments where the matrix or construct comprises the inorganic material together with a polymeric template, the excipient product under magnification, in certain embodiments has an appearance akin to a ball of string, wherein the string is entangled amongst itself to make the reticulated three-dimensional microstructure. The strands of the excipient product (i.e., the “strings” of the “ball”) are entangled in such a way as to create open spaces or pores between the entangled strands. The strands comprise the “primary structure” (hereinafter referred to as “primary walls”) of the matrix or construct, and the spaces or pore between the entangled strands are examples of “primary pores”. The surfaces of these strands are preferably not smooth, but rather have an irregular surface. In the circumstance where the pharmaceutical excipient product comprises a combination of the polymeric template material and the inorganic material, the primary walls may be comprised of the polymer coated or surrounded with the inorganic material. The primary walls may also be comprised of aggregates or agglomerates of inorganic materials in close proximity to (or in contact with) each other. In embodiments in which the polymeric template is removed after the inorganic material is coated (e.g., precipitated) onto its surface, the primary walls may be comprised of aggregates or agglomerates of inorganic materials in close proximity to (or in contact with) each other. In preferred embodiments, secondary particles or walls are formed on the surface of the primary walls. These secondary particles or walls are comprised of the inorganic material, which is preferably calcium phosphate or another pharmaceutically acceptable calcium salt. The spaces between the small crystals of the inorganic material are examples of “secondary pores” in accordance with the present invention. There is a third type of pore which may or may not be present in the pharmaceutical excipient product (constructs) of the present invention. This third type of pore (a “tertiary pore”) is a form of secondary pore which is not open to the surface of the construct (e.g., it is a hole or void formed by the connected interior surface of aggregated secondary particles). In situations where the strands comprising the primary walls of the construct are substantially smooth, there would be reduced amounts of secondary particles or walls in the construct. For this reason, one preferred way of forming an excipient in accordance with the invention is via the controlled crystal nucleation of the inorganic material onto the surface of a polymeric template in order to create a matrix or construct which comprises strands having an irregular surface, and thus comprising a sufficient degree of secondary walls to provide the construct with the desired specific surface area.

In certain preferred embodiments, the primary walls of the construct have a mean width (or thickness, as opposed to length) of from about 10 to about 500 μm, preferably from about 10 to about 200 μm, and in certain embodiments has a mean width from about 20 to about 100 μm. In theory, as thin a primary wall structure as possible is preferred, in such embodiments the mean width of the primary wall is from about 10 to about 50 μm.

In certain preferred embodiments, the mean width (or thickness, as opposed to length) of the secondary walls of the pharmaceutical excipient of the present invention are from about 0.01 to about 5 μm, and are preferably from about 0.5 to about 2 μm.

In certain preferred embodiments, the primary pore size is from about 5 to about 300 μm, in certain embodiments from about 10 to about 200 μm, and in certain preferred embodiments is from about 10 to about 50 μm, or 20 to 30 μm.

The secondary pores in the pharmaceutical excipients of the present invention have a mean size in the range from about 0.01 to about 5 μm, and preferably have a mean size range from about 0.01 to about 2 μm more preferably a mean size in the range from about 0.1 to about 3 μm, preferably from about 0.1 to about 2 μm.

It should be appreciated that the primary walls and secondary walls of the pharmaceutical excipients of the present invention are irregular in shape, and therefore it follows that the pores will be irregular in shape as well. Although it is possible for the excipient products (constructs) of the present invention to include secondary pores having a size smaller than 0.01 μm, it is presently believed that liquid penetration into such small pores will not be possible. Therefore, it is not believed that a drug will be able to be coated within such pores, and in turn it is believed that the dissolution medium (e.g., gastrointestinal fluid) will not be able to get into the pore to dissolve the drug if it was there. In preferred embodiments, the mean secondary pore size is from about 0.5 to about 1.5 μm across.

In a fourth aspect, the present invention provides a method of preparing a solid, reticulated matrix including the steps of, forming a reticulated template comprising an organic polymeric material, forming a construct comprising an aggregation of inorganic particles in association with said template, and solidifying said construct to form a solid, reticulated matrix comprising the inorganic particles in association with the inorganic polymeric material, where said matrix defines a plurality of pores with a mean width of 0.01-500 μm and/or has a specific surface area of at least 1 m²/g.

The reticulated template, aggregation of inorganic particles and the construct can be formed substantially simultaneously and the reticulated template can include the aggregation of inorganic particles or, alternatively, the inorganic particles can be coated onto the reticulated template.

In embodiments, the reticulated template can be formed by dispersing a second phase, that preferably comprises or consists essentially of solid particles or gas bubbles, in a liquid phase that comprises the organic polymeric material. In preferred embodiments, this liquid phase comprises a solution (that can be colloidal) of the organic polymeric material in a suitable solvent. Said solid particles are preferably formed from an organic material and, preferably, are soluble in a solvent in which the organic polymeric material, once set or solidified, is substantially insoluble. It is also preferred that the inorganic particles are also substantially insoluble in this latter solvent.

The reticulated template can be spontaneously formed from a solution of organic polymeric material in an appropriate solvent; or by the action of a cross-linking agent on a dissolved organic polymeric material.

In all cases, it is preferred for the reticulated template to be formed from a, preferably aqueous, solution of the polymeric material.

In embodiments, a proportion and, optionally, substantially all of the inorganic particles are formed by precipitation from a solution comprising the organic polymeric material. The inorganic particles, preferably, comprise an inorganic (ionic) salt and said solution, preferably, further comprises dissolved anions and/or cations of said salt. Preferably, before precipitation is initiated, this solution includes dissolved anions but substantially no dissolved cations, or dissolved cations and substantially no dissolved anions of the salt. Precipitation, preferably, is caused by the addition of a solution of the counter ions, in the salt, to the dissolved ions. Formation of the inorganic particles by precipitation can take place during or after the formation of the reticulated template.

In other embodiments, the inorganic particles are pre-formed and dispersed in a liquid phase which comprises the organic polymeric material. The latter is optionally in solution in a suitable solvent. Pre-formed inorganic particles can be included with others formed by in situ precipitation, and, when the reticulated template is formed by the step of dispersing solid particles or gas bubbles in a liquid phase that comprises the organic polymeric material, it is preferred that at least some and optionally all of the inorganic particles are pre-formed.

The inorganic particles are preferably crystalline.

The mean width of the particles or crystals of inorganic material is preferably in the range of about 0.1-50 μm. The mean width of the particles or crystals of inorganic material is preferably less than about 30, 25, 15, 10, 5 or 1 μm and, even more preferably, about 0.1-25, 0.2-10 or 0.2-5 μm. In some arrangements, the mean width of the particles or crystals of inorganic material can be between about 0.1 and about 0.2 μm, or between about 1 and about 15 μm.

In embodiments, the aggregation of inorganic particles can form a part of the reticulated template. The construct of reticulated template and aggregation of inorganic particles can be solidified by various means, including air-drying, spontaneous cross-linking, the action of a cross-linking agent, a temperature change, the act of forming the inorganic particles by precipitation, the influence of electro-magnetic radiation and/or any other known means for causing an organic polymeric materials to set into, or precipitate form solution as, a solid or substantially solid mass.

It is preferred that the inorganic particles comprise an alkaline earth metal carbonate or phosphate. It is also preferred that the method in accordance with this aspect of the present invention comprises forming an aqueous solution of an organic polymeric material and a soluble phosphate or carbonate salt and causing the alkaline earth metal carbonate or phosphate inorganic particles to precipitate from said solution by the addition of an aqueous solution of a salt of the alkaline earth metal. The preferred alkaline earth metal salts are the chlorides. The solution of alkaline earth metal salt can be added during or after the formation of the reticulated template.

In embodiments where the reticulated template is formed by entraining gas bubbles in a liquid phase comprising a solution of the organic polymeric material, it is preferred that some and possibly substantially all of the inorganic particles, especially when comprised of alkaline earth metal carbonate or phosphate, are caused to precipitate during said entrainment process. In embodiments wherein the reticulated structure is formed by distributing solid particles in a liquid phase comprising the organic polymeric material, the solid particles can be removed after the construct has been solidified by dissolution in an appropriate solvent.

Preferred organic polymeric materials include polysaccharides and proteins and the preferred alkaline metal earth salts are calcium carbonate and calcium phosphate. However, all of the organic polymeric materials and inorganic materials detailed in the foregoing discussion of other embodiments of this invention can be used in the practice of this fourth aspect of the invention. These materials can be utilised individually, or in admixture; that is to say, the organic polymeric material may comprise a mixture of polymers and the inorganic particles may include particles of more than one inorganic material. The preferred solid particles employed to form the reticulated template are latex particles, which can be removed from the finished porous matrix by the action of a solvent such as acetone. A mixture of organic polymeric materials can be employed and a mixture of alkaline and earth metal carbonates and phosphates may also be used.

In particularly preferred embodiments, methods in accordance with the fourth aspect of the invention can be employed to form pharmaceutical excipients, preferably pharmaceutical excipients in accordance with either the first, second or third aspect of the invention. In such preferred embodiments, the matrix formed by the method in accordance with the fourth aspect of the invention is the solid, reticulated matrix of the pharmaceutical excipient in accordance with the first, second or third aspect of the invention and, thus, can have any or all of the characteristics that a matrix of this latter type can have.

In a fifth aspect of the invention, a solid, reticulated matrix of organic polymeric material and inorganic particles is heated to a sufficiently high temperature to both eliminate the organic polymeric material and cause the inorganic particles to fuse together into a second solid, reticulated matrix defining a plurality of pores with a mean width of up to 100 μm.

Preferably, the organic polymeric material is eliminated and the organic particles caused to fuse by heating the solid, reticulated matrix to a temperature of at least about 800° C. and of up to about 1600° C., preferably, for a period of up to about 5 hours. The temperature of the solid, reticulated matrix, preferably, is raised from room temperature to said elevated temperature at a rate of between about 1 and about 20° C. per minute. The solid, reticulated matrix of organic polymeric material and inorganic particles, employed in the fifth aspect of this invention, can be a matrix as described above in the context of any of the aforementioned aspects of the invention. It can also be formed by any of the aforementioned methods for forming solid, reticulated matrices from organic polymeric material and inorganic particles.

When the solid, reticulated matrix is heated to a temperature of up to about 1100° C. and, preferably, up to about 1050° C., the resulting second solid, reticulated matrix comprises primary and secondary pores, wherein the primary pores have a mean width of about 2-500 μm and are defined between structural elements formed from the second matrix, the secondary pores have a mean width of 0.01-10 μm and are defined within said structural elements, and the mean width of the secondary pores is less than the mean width of the primary pores. Preferably, the mean width of the primary pores is at least about 5, 10, 20 or 40 μm and, preferably, no more than about 300, 200, 100 or 50 μm. It is preferred that the mean width of the secondary pores is at least about 0.01, 0.05, or 0.1 μm, and, preferably, no more than about 5, 3, 2, 1.5 or 1 μn. Preferably, at least about 50, 55, 70, 80, 90 or 95% of the primary pores have a width that is greater than the mean width of the secondary pores. It is also preferred for at least about 50, 55, 70, 80, 90 or 95% of the secondary pores to have a width which is less than the mean width of the primary pores. The structural elements can be in the form of primary walls that define the primary pores, and can comprise a network of secondary walls that define the secondary pores. The primary walls, preferably, have a mean width of about 10-500, 10-200, 20-100 or 10-50 μm. The secondary walls, preferably have a mean width of about 0.01-5 or 0.5-2 μm.

When the solid, reticulated matrix of organic polymeric material and inorganic particles is heated to a temperature in excess of 1100° C. and, preferably, to a temperature within the range of about 1200 or 1250-1500° C., the resulting second solid, reticulated matrix defines a plurality of pores with a mean width of about 0.01-50 μm. Preferably, the width of these pores is substantially normally distributed about their mean width with there being no discernable groupings of primary and secondary pores, or structural elements in the form of primary or secondary walls. The second solid, reticulated matrix, preferably, consists essentially or solely of said fused inorganic particles.

In a particularly preferred embodiment, the method in accordance with the fifth aspect of the invention comprises the steps of preparing a suspension of calcium carbonate or calcium phosphate particles in an aqueous solution of a polysaccharide, solidifying or setting said polysaccharide at a temperature of under about 100° C. and then sintering the resulting solid construct at a temperature of between about 900 and 1500° C. for up to about 5 hours to provide a solid, reticulated matrix in accordance with the third aspect of the invention. The polysaccharide, preferably, is dextran and can be set or solidified by air drying at room temperature or the addition or a cross linking agent, preferably epichlorhydrin.

In another particularly preferred embodiment, the method in accordance with the fourth aspect of the invention comprises the steps of preparing an aqueous solution of a polysaccharide and soluble phosphate or carbonate salt, admixing an aqueous solution of a calcium salt with said polysaccharide and phosphate solution, and allowing the resulting admixture to solidify. Preferably, said polysaccharide is dextran, xanthan gum or acacia gum. Preferably said phosphate or said carbonate is sodium phosphate or sodium carbonate and preferably said calcium salt is calcium chloride. The aqueous polysaccharide solution, preferably, contains at least about 40, 45, 50, 55, 60% polysaccharide and the phosphate or carbonate is preferably used in molar excess Preferably, the two solutions are admixed at a temperature in excess of about 50 or 60° C. whilst being stirred and, once admixed, the resulting admixture is allowed to cool to room temperature (about 20° C.) and allowed to solidify for a period of at least about 24 hours.

In a yet further preferred embodiment, the method in accordance with the fourth aspect of the invention comprises the steps of preparing an aqueous solution of a protein and a soluble carbonate or phosphate, entraining gas bubbles into the solution so as to form it into a foam, admixing an aqueous solution of a calcium salt with the phosphate and protein solution, and heating the resulting foam sufficiently to denature and solidify the protein. In an alternative embodiment, calcium carbonate or calcium phosphate particles are added to a protein solution, gas bubbles are then entrained within the slurry to form it into a foam, and the foam is then heated sufficiently to denature and solidify the protein. The gas bubbles are preferably air bubbles, the preferred protein is egg albumen and the foam is preferably heated to 40-100° C. for 12-24 hours.

In another preferred embodiment, the method in accordance with the fourth aspect of the invention comprises the steps of preparing an aqueous solution of a polysaccharide and phosphate or carbonate, dispersing beads of between about 0.1 and 10 μm in diameter in said solution, said beads being substantially insoluble in said solution, admixing into the resulting slurry an aqueous solution of a calcium salt; drying the resulting solid at a temperature of between about 15 and 80° C. and adding to said solid a solvent capable of dissolving said solid beads but not any substantial quantity of said resulting solid. The polysacchatide is preferably a gum and, more preferably, xanthan gum. The soluble phosphate or carbonate is preferably sodium phosphate or sodium carbonate. The solid beads are preferably formed from latex and dissolved out of the solid product by the addition of acetone. The soluble calcium salt is preferably calcium chloride and the aqueous polysaccharide solution, preferably includes between about 0.5 and 5% W/V of said polysaccharide.

In preferred embodiments, the inorganic constructs of the present invention can be prepared utilizing a pharmaceutically acceptable alkaline earth metal salt (such as calcium carbonate, and calcium phosphate), and are produced by controlled crystallization methods. Reticulated constructs can be prepared by controlling crystal nucleation with the aid of the pharmaceutically acceptable polymeric template, such dextran. Crystal nucleation preferably occurs along side the polymeric template, directing crystallization, to form thin strands (or filaments) of the construct. The polymeric template may be retained within the final construct or removed, by heating, as desired. In preferred embodiments of the present invention; the polymeric template is retained.

The invention is further directed in part to a method of preparing a pharmaceutical excipient, comprising dissolving a pharmaceutically acceptable polymeric material into a phosphate -or carbonate solution; adding calcium chloride to the solution containing the dissolved polymeric material in a controlled manner; and thereafter collecting the resultant solid material comprising a construct comprising calcium phosphate or carbonate crystals in intimate association with a polymer template, such that said construct has a specific surface area greater than about 10 m²/g. Preferably, the calcium chloride is added in a manner such that a porous matrix of a polymer template and said calcium phosphate or carbonate in crystalline form defining a construct is formed, said construct comprising strands defining primary walls having a mean width about 50 to about 500 μm, said primary walls comprised of (i) said polymer forming said polymeric template; (ii) aggregated crystals of said alkaline earth metal salt; and/or (iii) aggregated crystals of said alkaline earth metal salt coated on the surface of said polymer; said strands being arranged such that primary pores having a mean width from about 5 to about 300 μm are defined between at least two of said strands; said construct further comprising secondary walls extending from surfaces of said strands, said secondary walls having a mean width from about 0.01 to about 5 μm and comprising crystals of said alkaline earth metal salt; said secondary walls being arranged such that said construct includes secondary pores having a mean width from about 0.01 to about 5 μm defined between at least two secondary walls.

In further embodiments, the method comprises combining the pharmaceutical excipient of the invention with a therapeutic agent in a manner such that the therapeutic agent is coated onto the pharmaceutical excipient. Preferably, the therapeutic agent is coated onto the pharmaceutical excipient at a level, e.g., from about 1% to about 50% w/w. The therapeutic agent may be coated onto the surface of said pharmaceutical excipient via a method such as, e.g., solvent evaporation, freeze drying, and spray drying.

In further embodiments, the method further comprises incorporating the pharmaceutical excipient coated with the therapeutic agent into an oral solid dosage form.

In certain preferred embodiments of the present invention, the excipient or reticulated three-dimensional microstructures can be prepared by controlling crystal nucleation of an inorganic material (e.g., calcium phosphate, calcium carbonate and the like) with the aid of a pharmaceutically acceptable polymeric template (e.g., dextran, xanthan gum). Crystal nucleation occurs preferentially along the polymer template, directing crystallization, to form thin strands of the construct. The polymeric template may be retained within the final construct or remove by heating, as desired. In certain preferred embodiments, the polymeric template is retained to form part of the excipient or reticulated three-dimensional microstructure.

In other embodiments, pre-formed particles of inorganic material can be employed with or instead of particles form in situ. The use of pre-formed particles is especially preferred when the reticulated polymeric template is produced by a method involving a step of dispersing gas bubbles or solid particles within a liquid phase which comprises the organic polymeric material.

The pharmaceutical excipients of the present invention may be prepared by any method known to those skilled in the art. For example, one generalized method of manufacture is as follows: The pharmaceutically acceptable polymeric template material is dissolved into a phosphate or carbonate solution, then calcium chloride is added in a controlled manner. The calcium phosphate or carbonate crystals, for example, are caused to form (precipitate) in the presence of the template. The remaining liquid, if any, may be decanted off. Alternatively, the solid may be removed. Thereafter, the solid construct is dried.

For example, for the preparation of calcium phosphate structures with a construct, for example the following materials can be utilized: Na₂HPO₄: 2 moIL⁻¹, in the range of 0.1 to 10 moIL⁻¹. CaCl₂: 4 moIL⁻¹, in the range of 0.1 to 10 moIL⁻¹. Template Material: 25 t 85% w/w, in the range of 5 to 95% w/w. In this instance, % w/w is defined in relation to the weight of template material and the weight of calcium phosphate.

The polymeric material forming the template may then (preferably) be retained within the final construct or removed, e.g., by heating, as desired.

The excipient product (e.g., the construct) in many cases will not have well-defined geometry, but rather will comprise in irregular lump of viable material which can be readily broken up into smaller pieces (e.g., by tapping, grinding or milling). However, in any event, it is preferred that the step of breaking down the excipient product into smaller pieces be undertaken (if at all) in a manner which does not damage the structure of the product itself (i.e., does not destroy the three-dimensional reticulated microstructure of the material, including the pores or primary and secondary walls and pores).

It is preferred that the crystallization process described above be controlled such that the final crystal geometry possesses the highest surface area obtainable. The construct is preferably sieved or otherwise modified in order to provide particles of the end product pharmaceutical excipient which are useful to prepare, e.g., a solid oral dosage form. Preferably, the pharmaceutical excipient is sieved or otherwise broken down (while substantially maintaining the structure of the construct) such that the end product pharmaceutical excipient has a mean particle size from about 10 to about 500 μm, preferably from about 50 to about 300 μm, and in certain embodiments more preferably from about 100 to about 250 μm. The preferred size of the pharmaceutical excipient particles prepared in accordance with the invention will depend upon their end use, e.g., the type of oral solid dosage form to be manufactured.

Thus, in accordance with a sixth aspect of the present invention, there is provided a pharmaceutical excipient comprising a solid, reticulated matrix prepared or preparable by a method in accordance with the invention, preferably a method in accordance with the fourth and/or sixth aspects of the invention. Such a pharmaceutical excipient, preferably, is also a pharmaceutical excipient in accordance with the first, second and/or third aspect of the invention.

In embodiments of the sixth aspect of the invention, the pharmaceutical excipient comprises a solid, reticulated matrix in the form of a plurality agglomerations of organic polymeric material and inorganic particles or material, and is prepared or preparable by a method in which the reticulated template is formed by dispersing solid particles in a liquid phase that comprises the organic polymeric material. In preferred such embodiments, primary pores are located between adjacent such agglomerations and the solid particles employed to form the reticulated template are latex particles.

In a further preferred embodiment of this aspect of the invention, a pharmaceutical excipient in accordance of a third aspect of the invention is prepared or preparable by a method in accordance with the fifth aspect of the invention.

The invention is further directed, in an seventh aspect, to a pharmaceutical product comprising a pharmaceutical excipient in accordance with the invention and a pharmaceutically, preferably therapeutically, active agent.

The pharmaceutically active agent is preferably particulate and solid. In embodiments of the seventh aspect of the invention, the pharmaceutically active agent is intimately associated with the excipient. In such preferred embodiments, particles of the pharmaceutically active agent can be located within the pores of the solid, reticulated matrix and/or coated onto the matrix or excipient. Depending upon the size of the particles of pharmaceutically active agent, these can reside within the primary and/or secondary pores of the solid, reticulated matrix.

The pharmaceutically active agent preferably lies within Class 2 in the FDA adopted Biopharmaceutical Classification System (BCS). The pharmaceutically active agent can have an aqueous solubility of up to about 1 in 30 or 1 in 100 weight/volume, when measured at a temperature in the range of 15 to 25° C. The pharmaceutically active agent can be sparingly soluble to insoluble (as defined in Martindale).

Pharmaceutical products in accordance with the seventh aspect of the invention can include additional pharmaceutically acceptable excipients and/or diluents and can be oral solid dosage forms. Preferred oral solid-dosage forms include powders, capsules and tablets; capsules being the most preferred.

The pharmaceutically active agent is preferably crystalline. In preferred embodiments, the pharmaceutically active agent particles or crystals have a mean size or width of about 10 nm-10 μm, 10 nm-5 μm and, more preferably, less than about 1 μm. Preferably, the pharmaceutical products in accordance with the invention comprise from about 1 to about 50% W/W pharmaceutically active agent.

A purpose of the present invention is to facilitate entry of active agents into the bloodstream of persons needing the therapeutic effect of the active agent. Active agents suitable for use in the present invention include active agents of a biological nature and active agents of a chemical nature. The active agents of the present invention may also include pharmacological agents and therapeutic agents. Preferably the excipient of the present invention is useful with an active agent in an oral dosage form. In certain embodiments, the use of a reticulated microstructure or microstructures as provided by a pharmaceutical product according to the present invention can also be advantageous for use in aerosol administration which may be by way of nasal, oral or transdermal applications. The use of reticulated microstructures as provided by a pharmaceutical product according to the present invention can be advantageous for such aerosol administration at least partly due to the low mass density of such reticulated microstructures.

In certain embodiments, the therapeutic agent may typically comprise one or more biologically active materials suitable for oral administration.

In certain preferred embodiments, the therapeutic agent has an aqueous solubility of not greater than about 1 in 30 to 1 in 100, weight/volume, when measured at a temperature in the range of 15 to 25° C. Preferably, the pharmaceutical product comprises from about 1 to about 50% w/w with said therapeutic agent. Preferably, the pharmaceutical product has a mean particle size from about 10 to about 500 μm, in certain embodiments from about 50 to about 300 μm, and in yet other embodiments from about 100 to about 250 μm. The therapeutic agent may be coated onto said pharmaceutical excipient, e.g., via a process selected from the group consisting of solvent evaporation, spray drying and freeze drying.

The invention is further directed in part to an oral solid dosage form comprising a unit dose of the pharmaceutical product described herein. The oral solid dosage form may be in a form such as, e.g., a pharmaceutical powder, a capsule, or a tablet.

The present invention is further directed in part to a method of treatment, comprising administering an oral dosage form as described herein to a human patient. In a yet further aspect, the present invention provides an excipient or product in accordance with the invention for use in medicine (including veterinary medicine). Preferred such uses include the treatment of the human or animal body by therapy and diagnostic methods practised upon the human or animal body. The treatment may be prophylactic or may be in respect of an existing condition. Therapeutic (including prophylactic) and diagnostic methods, involving the use of an excipient or product in accordance with the invention, are also within the remit of the invention.

The use of such an excipients and products for the manufacture of medicaments for use in therapeutic or diagnostic methods to be practised on the human or animal body, lie within the scope of a further aspect of the invention.

According to the present invention, there is, therefore, provided a pharmaceutical product comprising at least one therapeutic agent and an excipient in accordance with the invention, whereby a unit dose of said therapeutic agent as provided by said pharmaceutical product can be administered to a patient during the passage of said therapeutic agent through the gastrointestinal tract of the patient, wherein said therapeutic agent is characterized as having an aqueous solubility of not greater than about 1 in 30 to 1 in 100, weight/volume, when measured at a temperature in the range of 15 to 25° C.

By virtue of the use of pharmaceutical excipients in accordance with the invention, the bio availability of poorly water-soluble drugs may be increased by increasing the rate of dissolution via one of two possible routes. The preferred option is to increase the surface area of the drug available for dissolution by presenting the drug coated- onto-the reticulated three-dimensional microstructure- of the excipient (matrix or construct). The drug may be coated onto the matrix or construct as multi-layers, or preferably a mono-layer, in order to generate maximum available surface area. The second option is to present the drug, as before, coated onto a high surface area excipient or construct in accordance with the invention which in this case will comprise a high surface area particle which may be spherical, cubic, or have assorted geometry. The particles can exhibit substantial surface roughness and may exhibit a high degree of porosity.

In a preferred embodiment of the present invention, the pharmaceutical composition includes an active agent which is not successfully orally administered, when dissolved or suspended in aqueous solution, wherein said active agent provides a therapeutic effect when administered to a patient by another means, and an effective amount of the pharmaceutical excipient of the invention to tender said active agent orally bioavailable. The active agent is preferably added to the pharmaceutical excipient of the invention in such a way that the active agent is coated onto the surface of the pharmaceutical excipient, such that the active agent is in intimate association with the pharmaceutical excipient of the invention.

As previously noted, therapeutic agents that can particularly benefit from use in pharmaceutical products according to the present invention include those typically defined as class 2 in the FDA adopted Biopharmaceutical Classification System (BCS). Such drug materials include those normally having an aqueous solubility of not greater than about 1 in 30 to 1 in 100, weight/volume, when measured at a temperature in the range of 15 to 25° C. Examples of sparingly soluble to insoluble (see page xiii of Martindale 31^{st} edition 1996. Published by The Royal Pharmaceutical Society) drugs that can be advantageously used in accordance with the present invention include, but are not limited to, the following: griseofulvin, acetaminophen (paracetamol), aspirin, atorvastatin, mefenamic acid, ibuprofen, ketoprofen, triamterene, naptoxen, theophylline, nifedipine, indomethacin, phenytoin, cyclosporine, acyclovir, alprazolam, allopurinol, acetohexamide, phenytoin, benzocaine, bendrofluazide, benzthiazide, betamethasone, chlorothiazide, cimetidine, carbamazepine, clofibrate, cozapine, cortisone acetate, cyclosporine, chlorthalidone, chlorpropamide, chlorpromazine, chlordiazepoxide, cyclopenthiazide, dexamethasone, diclofenac, digoxin, famotidine, fenprofen, fenbufen, flurbiprofen, fluoxitine, furosemide, gemfibrozil, glibenclamide, halopetidol, hydrochlorothiazide, hydrocortisone, hydroflumethiazide, ibuprofen, indomethacin, ketoprofen, lorazepam, lovastatin, methoxsalen, methylprednisone, naproxen, nifedipine, nitrofurantoin, nizatidine, oxazepam, oxyphenbutazone, omeprazole, oxyphenbutazone, phenylbutazone, piroxicam, phenytoin, pidolol, prednisone, pyrimethamine, phenindione, reserpine, spironolactone, trimethoprim, tactolimus, sulfisoxazole, sulfadiazine, temazepam, sulfamerazine, trioxsalen, pharmaceutically acceptable salts thereof, and the like.

Other drugs that are “sparingly soluble” to “insoluble” in water that may be used in accordance with the present invention and are listed in the refetence list and tables on the on pages 2071-2122 of U.S.P. XXIII, NF 18.

Active agents of a biological nature suitable for use in the present invention include, but are not limited to, proteins; polypeptides; peptides; hormones; polysaccharides, and particularly mixtures of muco-polysaccharides; carbohydrates; lipids; other organic compounds; and particularly compounds which by themselves do not pass (or which pass as only a fraction of the administered dose) through the gastro-intestinal mucosa and/or are susceptible to chemical cleavage by acids and enzymes in the gastro-intestinal tract; or any combination thereof. Further examples of active agents of a biological nature include, but are not limited to, the following, including synthetic, natural or recombinant sources thereof: growth hormones, including human growth hormones (hGH), recombinant human growth hormones (rhGH), bovine growth hormones, and porcine growth hormones; growth hormone-releasing hormones; interferons, including interleukin-1; interleukin-2; insulin, including porcine, bovine, human, and human recombinant, optionally having counter ions including sodium, zinc, calcium and ammonium; insulin-like growth factor, including IGF-1; heparin, including unfractionated heparin, heparinoids, dermatans, chondroitins, low molecular weight heparin, very low molecular weight heparin and ultra low molecular weight heparin; calcitonin, including salmon, eel, porcine and human; erythropoietin; atrial naturetic factor; antigens; monoclonal antibodies; somatostatin; protease inhibitors; adrenocorticotropin, gonadotropin releasing hormone; oxytocin; leutinizing-hotrmone-releasing-hormone; follicle stimulating hormone; glucocerebrosidase; thrombopoietin; filgrastim; prostaglandins; cyclosporin; vasopressin; cromolyn sodium (sodium or disodium chromoglycate); vancomycin; desferrioxamine (DFO); parathyroid hormone (PTH), including its fragments; antimicrobials, including anti-fungal agents; vitamins; analogs, fragments, mimetics or polyethylene glycol (PEG)-modified derivatives of these compounds; or any combination thereof.

Furthermore, pharmaceutical products according to the present invention may also exhibit advantageous flow properties that are often desirable in systems for aerosol administration, which may be by way of nasal, pulmonary or transdermal application.

By virtue of the use of pharmaceutical excipients prepared utilizing the reticulated three-dimensional microstructures of the invention, the bioavailability of poorly water-soluble drugs may be increased by increasing the rate of dissolution. Preferably the present invention increases the surface area of the drug available for dissolution by presenting the drug coated onto the reticulated three-dimensional microstructure of the matrix (construct). The drug may be coated onto the construct as multi-layers, or preferably a mono-layer, in order to generate maximum available surface area. In preferred embodiments, the drug is coated onto a high surface area construct which in this case would comprise a high surface area particle which may be spherical, cubic, or have assorted geometry. The particles will preferably exhibit substantial surface roughness and may be exhibit a high degree of porosity. The construct (excipient) preferably has a specific surface area greater than about 10 m²/g preferably from about 10 m²/g to about 40 m²/g. Preferably, increased particles stability reduces the tendency for particle agglomeration, (which would have the effect of greatly reducing the available surface area).

Excipients or inorganic constructs of the present invention can have a specific surface area of up to about 100 m²/g, or more preferably up to about 50 m²/g and preferred ranges of specific surface areas are from about 5 to about 50 mg²/g and more preferably from about 10 to about 40 m²/g. Alkaline earth-metal salts, such as calcium carbonate and calcium phosphate, have densities in the range of 2 to 3.5 g/cm³, several times greater than the majority of organic materials. The inorganic materials described for use within this technology possess very low specific surface areas, due to their high densities, when allowed to crystallize without undue influence. It is, therefore, of great importance to control the crystallization of these materials, when they are formed by in situ precipitation techniques, such that the final crystal geometry possesses the highest surface area obtainable. The easiest method of increasing the specific surface area of a material is to reduce the particle size. In order to achieve the desired surface area range, the particle size is preferably approximately from about 100 to about 200 nm.

Decreasing the particle size generates some concerns for secondary processing of excipients or constructs in accordance with the invention. As the effective particle size becomes smaller, the strength of the structure will decrease until it is too weak. It is important to find the balance between increased surface area and decreased structural strength. This balance can be, for example, calculated based on well known equations, based on particle geometry to model the desired physical characteristics of the particles.

In certain embodiments of the present invention, the pharmaceutical excipient product according to the present invention is utilized to prepare a pharmaceutical product comprising at least one therapeutic agent in crystalline form (which therapeutic agent is, for example, poorly water soluble). The pharmaceutical excipient product can comprise at least one reticulated three-dimensional microstructure comprising: a network of substantially interconnecting walls, said walls being provided by a multiplicity of crystals arranged to at least partially abut each other; and a multiplicity of pores defined by said substantially interconnecting walls. The reticulated three-dimensional microstructure may comprise an inorganic material (e.g., calcium phosphate), or construct comprising an inorganic material (e.g., calcium phosphate) and a template (e.g., a polymer such as dextran).

The walls of the reticulated microstructure, in certain embodiments, as provided by a pharmaceutical product according to the present invention have a thickness in the range of 0.01 to 500 μm, preferably 0.01 to 40 μm. In certain embodiments preferred wall thicknesses are dependent on the precise porous structure of the reticulated microstructure substantially as described herein.

According to a further preferred aspect of the present invention, there is provided, in certain embodiments, a pharmaceutical product comprising at least one therapeutic agent in crystalline form, said pharmaceutical product comprising a primary reticulated three-dimensional microstructure and a secondary reticulated three-dimensional microstructure, wherein said secondary reticulated microstructure defines the walls of said primary reticulated microstructure, and wherein said primary reticulated microstructure comprises: a network of substantially interconnecting primary walls, wherein said primary walls are provided by said secondary reticulated microstructure and substantially all of said primary walls have a thickness in the range of 10 to 40 μm; and a multiplicity of primary pores defined by said primary walls, wherein substantially all of the said primary pores have a pore size in the range of 40 μm to 60 μm; and said secondary reticulated microstructure comprises: a network of substantially interconnecting secondary walls, wherein said secondary walls are provided by a multiplicity of crystals arranged to at least partially abut each other substantially as hereinbefore described, wherein substantially all of said secondary walls have a thickness in the range of 0.5 to 5 μm; and a multiplicity of secondary pores defined by said secondary walls, wherein substantially all of said secondary pores have a pore size in the range of 0.1 to 5 μm.

In certain embodiments of the present invention, substantially all of the primary walls have a thickness in the range of about 10 to about 200 μm, preferably 20 to 30 μm. Furthermore, in certain embodiments of the present invention, substantially all of the primary pores have a pore size in the range about 5 to about 300 μm, preferably of 45 to 55 μm, such as about 50 μm. In certain embodiments of the present invention, substantially all of the secondary walls have a thickness in the range of about 0.01 to about 5 μm, preferably 0.5 to 1.5 μm. Furthermore, according to the above described further aspect of the present invention, substantially all of the secondary pores have a pore size in the range of about 0.01 to about 5 μm, preferably 0.5 to 1 μm.

A still further aspect of the above described alternative particularly preferred embodiment of the present invention provides a pharmaceutical product substantially as hereinbefore described wherein the crystals defining the interconnecting walls of the reticulated three-dimensional microstructure consist essentially of crystals of the above described physiologically acceptable support (excipient) and wherein crystals of the therapeutic agent are at least partially located within the pores of the reticulated microstructure substantially as hereinbefore described.

There is still further provided by the present invention use of at least one reticulated three-dimensional microstructure substantially as hereinbefore described as a physiologically acceptable support for crystals of a therapeutic agent, which reticulated microstructure comprises: a network of substantially interconnecting walls, said walls being provided by a multiplicity of crystals arranged to at least partially abut each other, said crystals defining said walls comprising crystals of a physiologically acceptable support material substantially as hereinbefore described; and a multiplicity of pores defined by said substantially interconnecting walls.

In certain preferred embodiments of the present invention, the multiplicity of crystals defining the walls of a reticulated microstructure or microstructures, as provided by a pharmaceutical product according to the present invention, comprise crystals of a physiologically acceptable support for therapeutic agent employed in a pharmaceutical product according to the present invention. Suitably the physiologically acceptable support is degradable in the physiological fluids of the gastrointestinal tract of a patient to yield physiologically acceptable degradation products.

More particularly, there is, therefore, provided by the above described preferred embodiment of the present invention, a pharmaceutical product comprising at least one therapeutic agent in crystalline form, said pharmaceutical product comprising at least one reticulated three-dimensional microstructure comprising: a network of substantially interconnecting walls provided by a multiplicity of crystals arranged to at least partially abut each other, said crystals defining said walls comprising crystals of a physiologically acceptable support for the therapeutic agent; and a multiplicity of pores defined by said substantially interconnecting walls.

In certain alternate embodiments of the present invention, there is provided a pharmaceutical product comprising at least one therapeutic agent, said pharmaceutical product comprising a reticulated three-dimensional microstructure, comprising: a network of substantially interconnecting walls, wherein said walls are provided by a multiplicity of crystals arranged to at least partially abut each other substantially as hereinbefore described, and wherein substantially all of said walls have a thickness of less than about 0.5 μm; and a multiplicity of pores defined by said walls, wherein substantially all of said pores have a pore size in the range of 0.1 to 1 μm. In such an embodiment of the present invention substantially all of the walls have a thickness in the range of 0.01 to 0.5 μm, preferably less than about 0.1 μm. Furthermore, in certain embodiments substantially all of the pores have a pore size typically in the range of 0.3 to 0.6 μm, more typically substantially all of the pores have a pore size of about 0.5 μm.

There is also provided in certain embodiments of the present invention a pharmaceutical product comprising at least one therapeutic agent in crystalline form, said pharmaceutical product comprising at least one reticulated three-dimensional microstructure comprising:

a network of substantially interconnecting walls, said walls being provided by a multiplicity of crystals arranged to at least partially abut each other; and

a multiplicity of pores defined by said substantially interconnecting walls;

wherein said reticulated microstructure has a specific surface area of at least 1 m²/g.

The pharmaceutical excipient of the present invention may be combined with the therapeutic agent in a manner such that the therapeutic agent is coated onto the excipient. This may be accomplished via any manner known to those skilled in the art including but not limited to solvent evaporation, freeze drying, and spray drying techniques known in the art.

The pharmaceutical excipient which is now coated with the therapeutic agent can thereafter be prepared into, e.g., an oral solid dosage form. For example, for the preparation of a powder containing a therapeutic agent, the pharmaceutical excipient can be coated to a level from about 1 to about 50% w/w with the therapeutic agent. In the case of capsules, the powder can then be mixed together with suitable amounts of a pharmaceutically acceptable lubricant and effective amounts of other optional pharmaceutically acceptable excipients, such as disintegrants, flow/packing promoters, etc., and thereafter the powder is filled into, e.g., a gelatin capsule of suitable size to contain a unit dose of the therapeutic agent.

For the production of tablets, the powder comprising the drug coated onto the pharmaceutical excipient (at a level, e.g., from about 1% to about 50% w/w), can be mixed together with an effective amount of a pharmaceutically acceptable lubricant, and further optional pharmaceutically acceptable excipients such as disintegrants, and diluents, and the mixture tablet according to methods well known to those skilled in the art.

When the final product to be manufactured is tablets, the complete mixture, in an amount sufficient to make a uniform batch of tablets, is then subjected to tableting in a conventional production scale tableting machine at normal compression pressure, i.e. about 2000-1600 lbs/sq in. However, the mixture should not be compressed to such a degree that there is subsequent difficulty in its hydration when exposed to gastric fluid. For best results, the tablets formed from the granulations of the present invention are from about 5 to about 20 kg hardness. The average flow of the pharmaceutical products prepared in accordance with the present invention is from about 25 to about 40 g/sec. In certain preferred embodiments, the tablets are formed using a lower than normal compression pressure, i.e., a pressure in the order of 15-35 Kg/cm² and preferably in the order of about 20-30 Kg/cm². The use of such lower compression pressures ensures that the reticulated micro-structure of the excipient in accordance with the invention is not damaged or distorted by the tableting process.

One of the limitations of direct compression as a -method of tablet manufacture is the size of the tablet. If the amount of active is high a pharmaceutical formulator may choose to wet granulate the active with other excipients to attain a decent size tablet with the right compact strength. Usually the amount of filler/binder or excipients needed in wet granulation is less than that in direct compression since the process of wet granulation contributes to some extent toward the desired physical properties of a tablet.

The average tablet size for round tablets is preferably about 300 mg to 750 mg and for capsule-shaped tablets about 700 mg to 1000 mg.

The oral dosage forms of the present invention, containing drug-coated excipient of the invention may include additional materials known to those skilled in the art as pharmaceutical excipients. Such pharmaceutical excipients include, for example, the following: Acidifying agents (acetic acid, glacial acetic acid, citric acid, fumaric acid, hydrochloric acid, diluted hydrochloric acid, malic acid, nitric acid, phosphoric acid, diluted phosphoric acid, sulfuric acid, tartaric acid); Alkalizing agents (strong ammonia solution, ammonium carbonate, diethanolamine, diisopropanolamine, potassium hydroxide, sodium bicarbonate, sodium borate, sodium carbonate, sodium hydroxide, trolamine); Anticaking agents (see glidant); Antifoaming agents (dimethicone, simethicone); Antimicrobial preservatives (benzalkonium chloride, benzalkonium chloride solution, benzelthonium chloride, benzoic acid, benzyl alcohol, butylparaben, cetylpyridinium chloride, chlorobutanol, chlorocresol, cresol, dehydroacetic acid, ethylparaben, methylparaben, methylparaben sodium, phenol, phenylethyl alcohol, phenyltnercuric acetate, phenylmetcuric nitrate, potassium benzoate, potassium sorbate, propylparaben, propylparaben sodium, sodium benzoate, sodium dehydroacetate, sodium propionate, sorbic acid, thimerosal, thymol); Antioxidants (ascorbic acid, acorbyl palmitate, butylated hydroxyanisole, butylated hydroxytoluene, hypophosphorous acid, monothioglycerol, propyl gallate, sodium formaldehyde sulfoxylate, sodium metabisulfite, sodium thiosulfate, sulfur dioxide, tocopherol, tocopherols excipient); Buffering agents (acetic acid, ammonium carbonate, ammonium phosphate, boric acid, citric acid, lactic acid, phosphoric acid, potassium citrate, potassium metaphosphate, potassium phosphate monobasic, sodium acetate, sodium citrate, sodium lactate solution, dibasic sodium phosphate, monobasic sodium phosphate); Chelating agents (edetate disodium, ethylenediaminetetraacetic acid and salts, edetic acid); Coating agents (sodium carboxymethylcellulose, cellulose acetate, cellulose acetate phthalate, ethylcellulose, gelatin, pharmaceutical glaze, hydroxypropyl cellulose, hydroxypropyl methylcellulose, hydroxypropyl methylcellulose phthalate, methacrylic acid copolymer, methylcellulose, polyethylene glycol, polyvinyl acetate phthalate, shellac, sucrose, titanium dioxide, carnauba wax, microcystalline wax, zein); Colorants (caramel, red, yellow, black or blends, ferric oxide); Complexing agents (ethylenediaminetetraacetic acid and salts (EDTA), edetic acid, gentisic acid ethanolmaide, oxyquinoline sulfate); Desiccants (calcium chloride, calcium sulfate, silicon dioxide); Emulsifying and/or solubilizing agents (acacia, cholesterol, diethanolamine (adjunct), glyceryl monostearate, lanolin alcohols, lecithin, mono- and di-glycerides, monoethanolanine (adjunct), oleic acid (adjunct), oleyl alcohol (stabilizer), poloxamer, polyoxyethylene 50 stearate, polyoxyl 35 castet oil, polyoxyl 40 hydrogenated castor oil, polyoxyl 10 oleyl ether, polyoxyl 20 cetostearyl ether, polyoxyl 40 stearate, polysorbate 20, polysotbate 40, polysorbate 60, polysorbate 80, propylene glycol diacetate, propylene glycol monostearate, sodium lauryl sulfate, sodium stearate, sorbitan monolaurate, soritan monooleate, sorbitan monopaimitate, sorbitan monostearate, stearic acid, trolamine, emulsifying wax); Filtering aids (powdered cellulose, purified siliceous earth); Flavors and perfumes (anethole, benzaldehyde, ethyl vanillin, menthol, methyl salicylate, monosodium glutamate, orange flower oil, peppermint, peppermint oil, peppermint spirit, rose oil, stronger rose water, thymol, tolu balsam tincture, vanilla, vanilla tincture, vanillin); Glidants and/or anticaking agents (calcium silicate, magnesium silicate, colloidal silicon dioxide, talc); Humectants (glycerin, hexylene glycol, propylene glycol, sorbitol); Plasticizers (castor oil, diacetylated monoglycerides, diethyl phthalate, glycerin, mono- and di-acetylated monoglycerides, polyethylene glycol, propylene glycol, triacetin, triethyl citrate); Polymers (e.g., cellulose acetate, alkyl celloloses, hydroxyalkylcelloloses, acrylic polymers and copolymers); Solvents (acetone, alcohol, diluted alcohol, amylene hydrate, benzyl benzoate, butyl alcohol, carbon tetrachloride, chloroform, corn oil, cottonseed oil, ethyl acetate, glycerin, hexylene glycol, isoptopyl alcohol, methyl alcohol, methylene chloride, methyl isobutyl ketone, mineral oil, peanut oil, polyethylene glycol, propylene carbonate, propylene glycol, sesame oil, water for injection, sterile water for injection, sterile water for irrigation, purified water); Sorbents (powdered cellulose, charcoal, purified siliceous earth); Carbon dioxide sorbents (barium hydroxide lime, soda lime); Stiffening agents (hydrogenated castor oil, cetostearyl alcohol, cetyl alcohol, cetyl esters wax, hard fat, paraffin, polyethylene excipient, stearyl alcohol, emulsifying wax, white wax, yellow wax); Suspending and/or viscosity-increasing agents (acacia, agar, alginic acid, aluminum monostearate, bentonite, purified bentonite, magma bentonite, carbomer 934p, carboxymethylcellulose calcium, carboxymethylcellulose sodium, carboxymethycellulose sodium 12, carrageenan, microcrystalline and carboxymethylcellulose sodium cellulose, dextrin, gelatin, guar gum, hydroxyethyl cellulose, hydroxypropyl cellulose, hydroxypropyl methylcellulose, magnesium aluminum silicate, methylcellulose, pectin, polyethylene oxide, polyvinyl alcohol, povidone, propylene glycol alginate, silicon dioxide, colloidal silicon dioxide, sodium alginate, tragacanth, xanthan gum); Sweetening agents (aspartame, dextrates, dextrose, excipient dextrose, fructose, mannitol, saccharin, calcium saccharin, sodium saccharin, sorbitol, solution sorbitol, sucrose, compressible sugar, confectioner=s sugar, syrup); Tablet binders (acacia, alginic acid, sodium carboxymethylcellulose, microcrystalline cellulose, dextrin, ethylcellulose, gelatin, liquid glucose, guar gum, hydroxypropyl methylcellulose, methycellulose, polyethylene oxide, povidone, pregelatinized starch, syrup); Tablet and/or capsule diluents (calcium carbonate, dibasic calcium phosphate, tribasic calcium phosphate, calcium sulfate, microcrystalline cellulose, powdered cellulose, dextrates, dextrin, dextrose excipient, fructose, kaolin, lactose, mannitol, sorbitol, starch, pregelatinized starch, sucrose, compressible sugar, confectioner=s sugar); Tablet disintegrants (alginic acid, microcrystalline cellulose, croscarmellose sodium, corspovidone, polacrilin potassium, sodium starch glycolate, starch, pregelatinized starch); Tablet and/or capsule lubricants (calcium stearate, glyceryl behenate, magnesium steatate, light mineral oil, polyethylene glycol, sodium stearyl fumatate, stearic acid, purified stearic acid, talc, hydrogenated vegetable oil, zinc stearate); Tonicity agent (dextrose, glycerin, mannitol, potassium chloride, sodium chloride); Vehicle: flavored and/or sweetened (aromatic elixir, compound benzaldehyde elixir, iso-alcoholic elixir, peppermint water, sorbitol solution, syrup, tolu balsam syrup); Vehicle: oleaginous (almond oil, corn oil, cottonseed oil, ethyl oleate, isoptopyl myristate, isopropyl palmitate, mineral oil, light mineral oil, myristyl alcohol, octyldodecanol, olive oil, peanut oil, persic oil, seame oil, soybean oil, squalane); Vehicle: solid carrier (sugar spheres); Vehicle: sterile (Bacteriostatic water for injection, bacteriostatic sodium chloride injection); Viscosity-increasing (see suspending agent); Water repelling agent (cyclomethicone, dimethicone, simethicone); and Wetting and/or solubilizing agent (benzalkonium chloride, benzethonium chloride, cetylpyridinium chloride, docusate sodium, nonoxynol 9, nonoxynol 10, octoxynol 9, poloxamer, polyoxyl 35 castor oil, polyoxyl 40, hydrogenated castor oil, polyoxyl 50 stearate, polyoxyl 10 oleyl ether, polyoxyl 20, cetostearyl ether, polyoxyl 40 stearate, polysorbate 20, polysorbate 40, polysorbate 60, polysotbate 80, sodium lauryl sulfate, sorbitan monolaureate, sorbitan monooleate, sorbitan monopalmitate, sorbitan monostearate, tyloxapol). This list is not meant to be exclusive, but instead merely representative of the classes of excipients and the particular excipients which may be used in oral dosage forms of the present invention.

An effective amount of any generally accepted pharmaceutical lubricant, including calcium or magnesium soaps is preferably added to the mixture of ingredients (including medicament) prior to compression of the drug coated excipient into oral solid dosage forms, such as tablets. An example of a suitable lubricant is magnesium stearate in an amount of about 0.5 to about 3% by weight of the solid dosage form. An especially preferred lubricant is sodium stearyl fumarate, NF, commercially available under the trade name Pruv7 from Penwest Pharmaceuticals Co.

Direct compression diluents are widely used in the pharmaceutical arts, and may be used to manufacture oral solid tablets containing the drug coated excipient of the invention. Such direct compression diluents may be obtained from a wide variety of commercial sources. Examples of such pre-manufactured direct compression excipients include Emcocel⁷ (microcrystalline cellulose, N.F.), Emdex⁷ (dextrates, N.F.), and Tab-Fine⁷ (a number of direct-compression sugars including sucrose, fructose and dextrose), all of which are commercially available from Penwest Pharmaceuticals Co., Patterson, N.Y.). Other direct compression diluents include Anhydrous lactose (Lactose N.F., anhydrous direct tableting) from Sheffield Chemical, Union, N.J. 07083; Elcems⁷ G-250 (powdered cellulose), N.F.) from Degussa, D-600 Frankfurt (Main) Germany; Fast-Flo Lactose⁷ (Lactose, N.F., spray dried) from Foremost Whey Products, Banaboo, Wis. 53913; Maltrin⁷ (Agglomerated maltodextrin) from Grain Processing Corp., Muscatine, Iowa 52761; Neosorb 60⁷ (Sorbitol, N.F., direct-compression from Roquette Corp., 645 5th Ave., New York, N.Y. 10022; Nu-Tab⁷ (Compressible sugar, N.F.) from Ingredient Technology, Inc., Pennsauken, N.J. 08110; Polyplasdone XL⁷ (Crospovidone, N.F., cross-linked polyvinylpyrrolidone) from GAF Corp., New York, N.Y. 10020; Primojel⁷ (Sodium starch glycolate, N.F., carboxymethyl starch) from Generichem Corp., Little Falls, N.J. 07424; Solka Floc⁷ (Cellulose floc) from Penwest Pharmaceuticals Co., Patterson, N.Y. 10512; Spray-dried lactose⁷ (Lactose N.F., spray dried) from Foremost Whey Products, Baraboo, Wis. 53913 and DMV Corp., Vehgel, Holland; and Sta-Rx 1500⁷ (Starch 1500) (Pregelatinized starch, N.F., compressible) from Colorcon, Inc., West Point, Pa. 19486.

In the case where a pharmaceutical product according to the present invention comprises paracetamol, it can be for use in the manufacture of a medicament for the treatment of pain.

Pharmaceutical products according to the present invention are particularly suitable for oral administration, although the most suitable route will generally depend upon the condition of a patient and the disease being treated. Indeed pharmaceutical products provided by the present invention can also be particularly suitable as aerosols, whether for nasal, oral or dermal administration. The precise amount of a pharmaceutical product according to the present invention to be administered to a patient will be the responsibility of an attendant physician, although the dose employed will depend upon a number: of factors, including the age and sex of the patient, the specific disease being treated and the route of administration.

In certain embodiments, the products of the present invention may be used in a drug delivery system for delivery of a drug for gastrointestinal deposition. Such a system can comprise a multiple unit dosing device containing multiple unit doses of a product as described herein, the device upon actuation delivering a unit dose of the product for gastrointestinal deposition, the pharmaceutical product of the invention having a mean particle size of greater than 10 μm in order to minimize pulmonary deposition of the multiparticulates of the formulation and less than about lmm such that an effective dose of the drug cannot be delivered into the lower lung of a human patient. The drug delivery system can be used to administer the unit dose of the product into the oral cavity of the patient (in-vivo) or to dispense the unit dose into an intermediate receptacle (ex-vivo) for subsequent gastrointestinal deposition. Oral drug delivery systems and devices for oral powders are disclosed in WO 01/64812 and PCT/IB02/03590, both entitled “Improvements In Or Relating To The Delivery Of Oral Drugs”; and U.S. Provisional Application No. 60/362,307 entitled “Drug Storage and Delivery Devices” filed Mar. 7, 2002; the disclosures of which are hereby incorporated by reference in their entireties for all purposes. In certain embodiments, the products of the present invention may be used to provide a method of preparing a drug delivery system for delivering multiple doses of a drug for gastrointestinal deposition, comprising preparing a product as described herein in a manner wherein the product, when placed in the oral cavity, is not deposited in any substantial amount in the lungs; and placing multiple unit doses of the formulation in a device which meters a single unit dose at a time for delivery.

Finally, there is further provided by the present invention a method of treating a disease, which method comprises administering to a patient a therapeutically effective amount of a pharmaceutical product according to the present invention.

Where a plurality of alternative values, ranges and range end points are given in this specification, each one should be considered as individually preferred and, hence, as being an individual preferred embodiment or feature of the invention. Moreover, it should be understood that, unless the contrary is clearly impossible, each and every possible combination of such values should be understood to have been individually disclosed by this specification. It should also be understood that the physical and chemical properties of pharmaceutical excipients in accordance with the invention are such as to render them suitable for use in preparing pharmaceutically acceptable products and, preferably, for use in preparing orally administered pharmaceutical products.

### BRIEF DESCRIPTION OF THE FIGURES

FIG. 1 is an SEM (scanning electron microscope) image of Example 1 taken at ×70 magnification which illustrates the primary structure present within the material. Examples of primary pores are indicated in the figure.

FIG. 2 is an SEM image of Example 1 taken at ×60 magnification which illustrates the primary structure present within the material. Examples of primary wall thicknesses are indicated in the figure.

FIG. 3 is an SEM image of Example 1 taken at ×1400 magnification which illustrates that the primary particles have an irregular geometry and a smooth surface. FIG. 3 also illustrates that packed between the larger primary particles are approximately 1 μm sized particles that define a secondary structure. Examples of primary particles are indicated in the figure.

FIG. 4 is an SEM image of Example 2 taken at ×50 magnification which illustrates the primary wall structure present within the material. The Primary walls are observed in the range of 50-500 μm defining a primary pore size in the range of 10-500 μm. Examples of primary pores and primary wall thicknesses are indicated in the figure.

FIG. 5 is an SEM image of Example 2 taken at ×270 magnification which illustrates the composition of the primary walls in more detail than FIG. 4. Primary particles are observed falling into two size distributions. Large particles in the range of 10-50 μm and small particles in the range of 0.5-5 μm are present. Examples of primary wall thickness is indicated in the figure.

FIG. 6 is an SEM image of Example 2 taken at ×1300 magnification and supports the findings of FIG. 5. A high surface roughness is observed in FIG. 6 along with a secondary pore structure of pores in the range of 0.1-1 μm. Examples of secondary pores and secondary particles are indicated in the figure.

FIG. 7 is an SEM image of Example 3 taken at ×220 magnification which illustrates the primary wall structure present within the material. Primary walls are observed in the range of 50-200 μm defining a primary pore size in the range of 10-100 μm. The primary wall structure comprises of particles that are smooth, of irregular geometry and in the range of 1-30 μm. Examples of primary pores and primary wall thicknesses are indicated in the figure.

FIG. 8 is an SEM image of Example 3 taken at ×650 magnification and illustrates the composition of the primary walls in more detail. A secondary pore structure is observed in the range of 0.1-1 μm, formed as the interstitial spacing between primary particles as opposed to spacing between a smaller secondary particle. Examples of secondary pores are indicated in the figure.

FIG. 9 is an SEM image of Example 4 taken at ×1500 magnification and illustrates the primary wall structure present within the material. Primary walls are observed in the range of 10-50 μm defining a primary pore size in the range of 5-20 μm. The primary wall structure comprises of particles that are ‘fluffy’ in appearance, of irregular geometry and in the range of 1-10 μm. Examples of primary wall thickness and a primary pore are indicated in the figure.

FIG. 10 is an SEM image of Example 4 taken at ×10000 magnification and illustrates the composition of the primary walls in more detail. The primary particles consist of a combination of small particles in the range of less than 1 μm and rod needle shaped particles approximately 100 nm thick and 0.1-2 μm long. These secondary particles define a secondary pore structure in the range of less than or equal to 1 μm. Examples of secondary pores and secondary particles are indicated in the figure.

FIG. 11 depicts absorbance vs. time curve results for the dissolution test of the nifedipine coated construct of Example 12.

FIG. 12 depicts absorbance vs. time curve results for the dissolution test of the USP grade nifedipine of Example 12.

FIG. 13 is an SEM image taken at ×180 magnification and shows the material prepared in Example 13a. This material comprises clusters of calcium phosphate incorporating spherical pores of 300 nm diameter. The individual clusters abut each other to form a continuous structure defining a primary pore structure in the range of 2-50 μm.

FIG. 14 is an SEM image of a material prepared using the method described in Example 13, taken at ×800 magnification. This figure shows a single-cluster of calcium phosphate prepared using approximately 2 μm latex particles.

FIG. 15 is an SEM image of a material prepared using the method described in Example 14, taken at ×40 magnification. This figure shows a reticulated structure prepared using pre-formed calcium carbonate particles and shows these particles to be located within the interstitial spaces of the foam and at the interface of each air bubble.

FIG. 16 is an SEM image of the material shown in FIG. 15, but taken at ×300 magnification, showing one of the bubbles depicted in FIG. 15.

FIG. 17 is an SEM image taken at ×5,000 magnification of the wall formed between two of the bubbles in the material shown in FIGS. 15 and 16.

FIG. 18 is an SEM image of a material prepared by the in situ precipitation method described in Example 14, at ×1000 magnification. The Figure shows near-spherical particles of calcium carbonate, in the range of 1-15 μm in diameter orientated along the interface of each air bubble:

FIG. 19 is an SEM image of the material shown in FIG. 18, but at ×2,500 magnification and shows the lose packing of particles along the interface between two air bubbles.

FIG. 20 is an SEM image taken at ×220 magnification and showing a reticulated structure prepared using pre-formed calcium phosphate particles and heating to a temperature of 1,000° C. in the method described in Example 15.

FIG. 21 is an SEM image of a single strand of the structure shown in FIG. 20, taken at ×4,000 magnification, and shows the strand to consist of sintered particles and sub-micron sized pores.

FIG. 22 is an SEM image of a material prepared using the method described in example 15, in which the matrix was heated to a temperature of 1,350° C., at ×200 magnification.

FIG. 23 is an SEM image of the material shown in FIG. 22, taken at ×350 magnification.

FIG. 24 is an SEM image of a portion of the structure shown in FIG. 23, but at ×1,000 magnification, showing minimal grain boundaries and confirming the effect of sintering.

The present invention will now be further illustrated by the following Examples, which do not limit the scope of the invention in any way.

### EXAMPLE 1

Preparation of Calcium Phosphate Structures with a Template (Forming a Construct)

In Example 1, 55% w/w dextran was added to 7 mL of a 2 M Na₂HPO₄ aqueous solution. The solution was warmed in a water bath to 70° C. and stirred vigorously to promote dissolution of the polymer. Following complete dissolution of the dextran the solution became clear. At this stage 1 mL of a 4 M CaCl₂ aqueous solution was added dropwise to the reaction vessel. Stirring was then ceased and the vessel removed from the water bath and allowed to cool to room temperature. The sample was left for 48 hours to allow the contents of the reaction vessel to solidify.

A small fraction of the solid material was carefully broken off of the bulk sample, placed onto a metal stub and sputter coated with gold in preparation for imaging using a Jeol 5600 scanning electron microscope. Images were recorded at several magnifications to determine the existence of both primary and secondary level structure.

Specific surface area was measured using a BET method on a Coulter SA3100. Approximately 1 gram of material was crushed in a controlled manner, such that the crushed particle size was sufficient that the sample would enter the measuring chamber. This measurement was repeated three times to ensure repeatability.

Scanning Electron Microscope Images of Example 1:

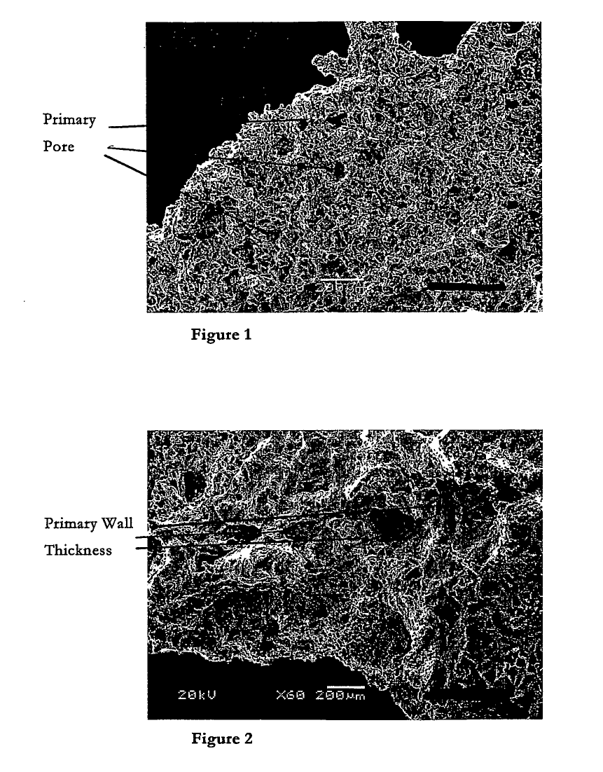

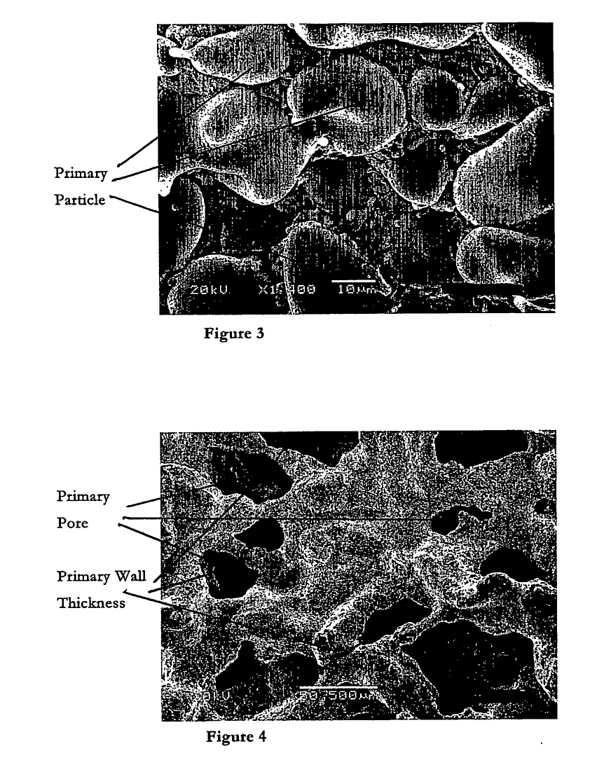

FIG. 1 is taken at ×70 magnification and FIG. 2 is taken at ×60 magnification. Both images illustrate the primary structure present within the material. It is observed that the material consists of a primary wall structure constructed, predominantly, from irregularly shaped particles in the range of 1-100 μm. The primary wall structure defines a primary pore structure in the range of 10-200 μm. FIG. 3 is taken at ×1400 magnification. At this magnification the primary particles can clearly be identified as having an irregular geometry and a smooth surface. Packed between the larger primary particles are approximately 1 μm sized particles that define a secondary structure. Very few pores are observed within the secondary structure, those that are present are of the order of 1 μm in size. BET adsorption measurements found the specific surface area to be 0.4 m²g⁻¹.

The particles that define the primary wall structure are both large and exhibit very low surface roughness. These two factors combined lead to the relatively low specific surface area measured for this sample. However, the material possesses the open structure required to stabilize poorly water soluble drugs and may be useful with low dosage compounds. Although a secondary structure exists within this material, the inaccessible nature of the pores (small pore size) would prevent this material from being useful as the sole excipient in a tableted solid dosage form (i.e., without the inclusion of additional (compressible) pharmaceutical excipients).

### EXAMPLE 2

Preparation of Calcium Phosphate Structures with a Template (Forming a Construct)

In Example 2, 69% w/w xanthan gum was added to 7 mL of a 2 M Na₂HPO₄ aqueous solution. The solution was warmed in a water bath to 70° C. and stirred vigorously to promote dissolution of the polymer. Following complete dissolution of the xanthan gum the solution became clear. At this stage 1 mL of a 4 M CaCl₂ aqueous solution was added dropwise to the reaction vessel. Stirring was then ceased and the vessel removed from the water bath and allowed to cool to room temperature. The sample was left for 24 hours to allow the contents of the reaction vessel to solidify.

A small fraction of the solid material was carefully broken off of the bulk sample, placed onto a metal stub and sputter coated with gold in preparation for imaging using a jeol 5600 scanning electron microscope. Images were recorded at several magnifications to determine the existence of both primary and secondary level structure.

Specific surface area was measured using a BET method on a Coulter SA3100. Approximately 1 gram of material was crushed in a controlled manner, such that the crushed particle size was sufficient that the sample would enter the measuring chamber. This measurement was repeated three times to ensure repeatability.

Scanning Electron Microscope Images of Example 2:

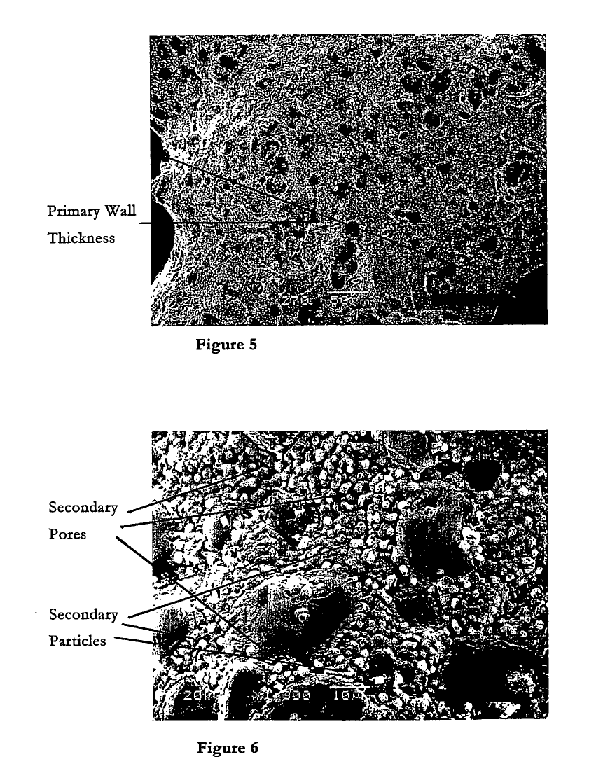

FIG. 4 is taken at ×50 magnification and illustrates the primary wall structure present within the material. Primary walls are observed in the range of 50-500 μm defining a primary pore size in the range of 10-500 μm. FIG. 5 is taken at ×270 magnification and illustrates the composition of the primary walls in more detail. Primary particles are observed falling into two size distributions. Large particles in the range of 10-50 μm and small particles in the range of 0.5-5 μm are present. FIG. 6 is taken at ×1300 magnification and supports the findings of FIG. 5. A high surface roughness is observed in FIG. 6 along with a secondary pore structure of pores in the range of 0.1-1 μm. BET adsorption measurements found the specific surface area to be 2.1 m²g⁻¹.

The primary wall structure is observed, at high magnification, to consist of particles in the range of 0.5-50 μm that define a secondary pore structure of pores in the order of less than 1 μm. Although the contribution of the secondary structure to the specific surface area is significant, the specific surface area of the material is lower than expected due to the size of the primary wall structure. This material possesses the open structure required to stabilize poorly water soluble drugs and may be useful in powder delivery, capsules and tablets of low dosage compounds.

### EXAMPLE 3

Preparation of Calcium Phosphate Structures with a Template (Forming a Construct)

In Example 3, 69% w/w acacia gum was added to 7 mL of a 2 M Na₂HPO₄ aqueous solution. The solution was warmed in a water bath to 70° C. and stirred vigorously to promote dissolution of the polymer. Following complete dissolution of the acacia gum the solution became cleat. At this stage 1 mL of a 4 M CaCl₂ aqueous solution was added dropwise to the reaction vessel. Stirring was then ceased and the vessel removed from the water bath and allowed to cool to room temperature. The sample was left for 24 hours to allow the contents of the reaction vessel to solidify.

A small fraction of the solid material was carefully broken off of the bulk sample, placed onto a metal stub and sputter coated with gold in preparation for imaging using a Jeol 5600 scanning electron microscope. Images were recorded at several magnifications to determine the existence of both primary and secondary level structure.

Specific surface area was measured using a BET method on a Coulter SA3100. Approximately 1 gram of material was crushed in a controlled manner, such that the crushed particle size was sufficient that the sample would enter the measuring chamber. This measurement was repeated three times to ensure repeatability.

Scanning Electron Microscope Images of Example 3:

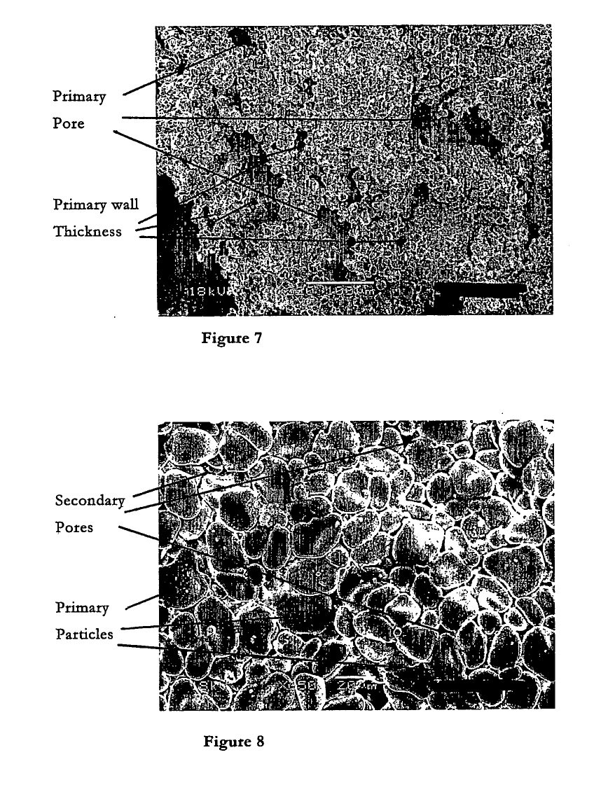

FIG. 7 is taken at ×220 magnification and illustrates the primary wall structure present within the material. Primary walls are observed in the range of 50-200 μm defining a primary pore size in the range of 10-100 μm. The primary wall structure comprises of particles that are smooth, of irregular geometry and in the range of 1-30 μm. FIG. 8 is taken at ×650 magnification and illustrates the composition of the primary walls in more detail. A secondary pore structure is observed in the range of 0.1-1 μm, formed as the interstitial spacing between primary particles as opposed to spacing between a smaller secondary particle.

BET adsorption measurements found the specific surface area to be 0.2 m²g⁻¹.

The primary wall structure is observed, at high magnification, to consist of particles in the range of 1-30 μm that define a secondary pore structure of pores in the order of less than 1 μm. Although the contribution of the secondary structure to the specific surface area is significant, the specific surface area of the material is lower than expected due to the size of the primary wall structure. This material possesses the open structure required to stabilize poorly water soluble drugs and my be useful in powder delivery, capsules and tablets of low dosage compounds.

### EXAMPLE 4

Preparation of Calcium Phosphate Structures with a Template (Forming a Construct)

In Example 4, 5% w/w xanthan gum was added to 500 mL of a 2 M Na₂HPO₄ aqueous solution. To this solution 5% w/w calcium carbonate was added as an insoluble bulking agent. The solution was warmed in a water bath to 70° C. and stirred vigorously to promote dissolution of the polymer. Following complete dissolution of the xanthan gum the solution became clear. At this stage 100 mL of a 4 M CaCl₂ aqueous solution was added dropwise to the reaction vessel. Stirring was then ceased and the vessel removed from the water bath and allowed to cool to room temperature. Excess solvent was then decanted from the reaction vessel and the solid sample was placed under vacuum at 60° C. for 96 hours to allow the solid to dry.

A small fraction of the solid material was carefully broken off of the bulk sample, placed onto a metal stub and sputter coated with gold in preparation for imaging using a Jeol 6310 scanning electron microscope. Images were recorded at several magnifications to determine the existence of both primary and secondary level structure.

Specific surface area was measured using a BET method on a Coulter SA3100. Approximately 1 gram of material was crushed in a controlled manner, such that the crushed particle size was sufficient that the sample would enter the measuring chamber. This measurement was repeated three times to ensure repeatability.

Scanning Electron Microscope Images of Example 4:

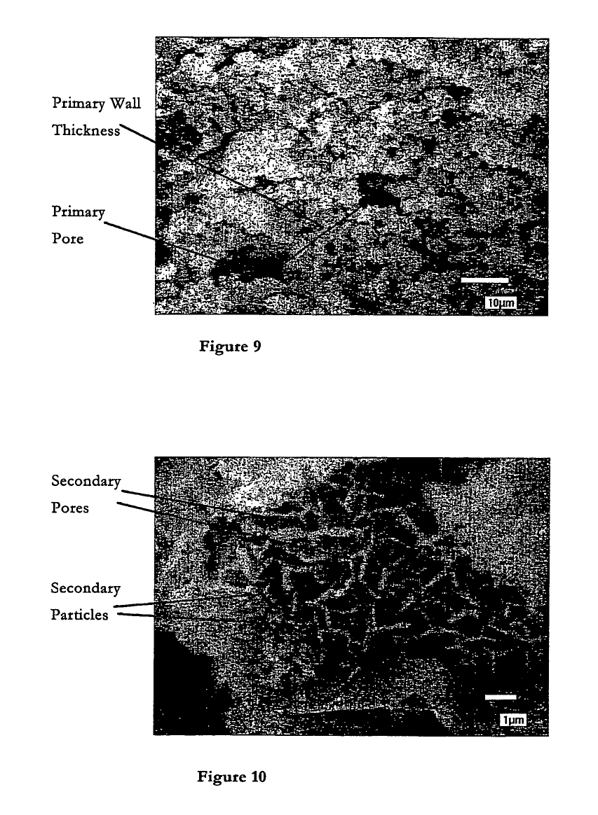

FIG. 9 is taken at ×1500 magnification and illustrates the primary wall structure present within the material. Primary walls are observed in the range of 10-50 μm defining a primary pore size in the range of 5-20 μm. The primary wall structure comprises of particles that are ‘fluffy’ in appearance, of irregular geometry and in the range of 1-10 μm. FIG. 10 is taken at ×10000 magnification and illustrates the composition of the primary walls in more detail. The primary particles consist of a combination of small particles in the range of less than 1 μm and rod needle shaped particles approximately 100 nm thick and 0.1-2 μm long. These secondary particles define a secondary pore structure in the range of less than or equal to 1 μm. BET adsorption measurements found the specific surface area to be 13.4 m²g⁻¹.

### EXAMPLE 5

Applying Drug to a Construct via Solvent Evaporation

In Example 5, the construct of Examples 1-4 are taken and passed through a sieve stack to collect a size fraction in the range 50-500 μm. 2.7 g of the appropriate size fraction are added to a solution containing 300 mg of Nifedipine dissolved in 1-5 mL ethanol (analytical grade) in an amber glass reaction vessel. The slurry is then sonicated in an ultrasonic bath for 10 minutes to ensure complete wetting of the solid. The reaction vessel is then transferred to a vacuum oven where it is placed under vacuum and heated to between 30-50° C. for up to 24 hours to ensure complete removal of the ethanol.

Coating of the constructs of Examples 1-4 are confirmed by HPLC and USP type IV dissolution testing.

### EXAMPLE 6

Applying Drug to a Construct via Freeze Drying

In Example 6, the construct of Examples 1-4 are taken and passed through a sieve stack to collect a size fraction in the range 50-500 μm. 2.7 g of the appropriate size fraction ate added to a solution containing 300 mg of Nifedipine dissolved in 1-5 mL ethanol (analytical grade) in an amber glass reaction vessel. The slurry is then sonicated in an ultrasonic bath for 10 minutes to ensure complete wetting of the solid. The reaction vessel is then transferred to a crystallising dish where it is placed into a Virtis Advantage freeze drying unit. The sample is held at a temperature below its freezing point for 3 hours, held at its freezing point for 3 hours and finally held at room temperature to ensure complete removal of the ethanol.

Coating of the constructs of Examples 1-4 are confirmed by HPLC and USP type IV dissolution testing.

### EXAMPLE 7

Applying Drug to a Construct via Spray Drying

In Example 7, the construct of Examples 1-4 are taken and passed through a sieve stack to collect a size fraction in the range 50-500 μm. 27 g of the appropriate size fraction is added to a solution containing 3 g of Nifedipine dissolved in 10-50 mL ethanol (analytical grade) in an amber glass reaction vessel. The slurry is then sonicated in an ultrasonic bath for 10 minutes to ensure complete wetting of the solid. The contents of the vessel are then pumped through a Buchi mini spray dryer at between 1-5 mLmin-1 at 90° C., resulting in particles of coated construct.

Coating of the constructs of Examples 1-4 ate confirmed by HPLC and USP type IV dissolution testing.

### EXAMPLE 8

Production of Solid Dosage Form (Powder)

In Example 8, the drug-coated construct prepared in any of Examples 5-7 is used to prepare an oral solid dosage form (in this case, e.g., a powder). In the powder dosage form, the drug-coated construct (coated at between 1-50% w/w) is delivered directly as a powder.

### EXAMPLE 9

Production of Solid Dosage Form (Capsules)

In Example 9, the drug-coated construct prepared in any of Examples 5-7 is used to prepare an oral solid dosage form (in this case, e.g., a capsule). In the capsule dosage form, the following ingredients listed below are mixed together and filled into capsules.

0.25% w/w lubricant, e.g. Magnesium stearate;

<3% w/w disintegrant, e.g. Sodium starch glycolate (if necessary); requisite amount of Flow/packing promoter, e.g. Starch 15 (if necessary); and requisite amount of coated construct (coated at between 1-50% w/w) (concentrations of diluent and coated construct will vary with drug loading concentration).

### EXAMPLE 10

Production of Solid Dosage Form (Large Tablets)

In Example 10, the drug-coated construct prepared in any of Examples 5-7 is used to prepare an oral solid dosage form (in this case, e.g., a large tablet >250 mg tablet mass, self compressing tablet with no diluent). In the large tablet form, the following ingredients listed below are mixed together and compressed into a tablet using a tablet press.

0.5% w/w lubricant e.g. Magnesium stearate;

3% w/w cdisintegrant e.g. Sodium starch glycolate; and

96.5% w/w coated construct (coated at between 1-50% w/w).

### EXAMPLE 11

Production of Solid Dosage Form (Small Tablets)

In Example 11, the drug-coated construct prepared in any of Examples 5-7 is used to prepare an oral solid dosage form (in this case, e.g., a small tablet <250 mg tablet mass.)). In the small tablet form, the following ingredients listed below are mixed together and compressed into a tablet using a tablet press.

0.5% w/w lubricant e.g. Magnesium stearate;

3% w/w disintegrant e.g. Sodium starch glycolate;

requisite amount of diluent e.g. Microcrystalline cellulose or Lactose; and

requisite amount of coated construct (coated at between 1-50% w/w) (concentrations of diluent and coated construct will vary with drug loading concentration).

### EXAMPLE 12

In Example 12, a construct prepared in accordance with Example 4 was passed through a sieve stack to collect a size fraction in the range 300-600 μm. 95% w/w of the construct was added to 10 mL of a 0.03 moIL⁻¹ nifedipine in ethanol solution. The slurry was placed into an ultrasound bath and sonicated for 10 minutes to ensure complete wetting of the solid. The sample vessel was then warmed in an 80° C. water bath until all of the ethanol had evaporated. The sample vessel was then transferred to an oven and heated at 60° C. for 30 minutes to ensure the sample was completely dry.

Coating of the construct was confirmed by HPLC and USP type IV dissolution testing. HPLC an USP type IV dissolution testing results were as follows for the formulation of Example 12:

HPLC

Content uniformity was determined by HPLC using a Jones Genesis column (C18 4 μm, 150×4.6 mm ID). The mobile phase was 50:40:10 deionised water:methanol:acetonitrile. 30 mg samples of the nifedipine coated excipient were dissolved in 50:50 acetonitrile: deionised water. 10 μL injections were made and the resulting peaks compared to a nifedipine USP standard of known concentration.

It was found that in all cases the nifedipine concentration was 2.5±0.5% w/w.

Dissolution Data

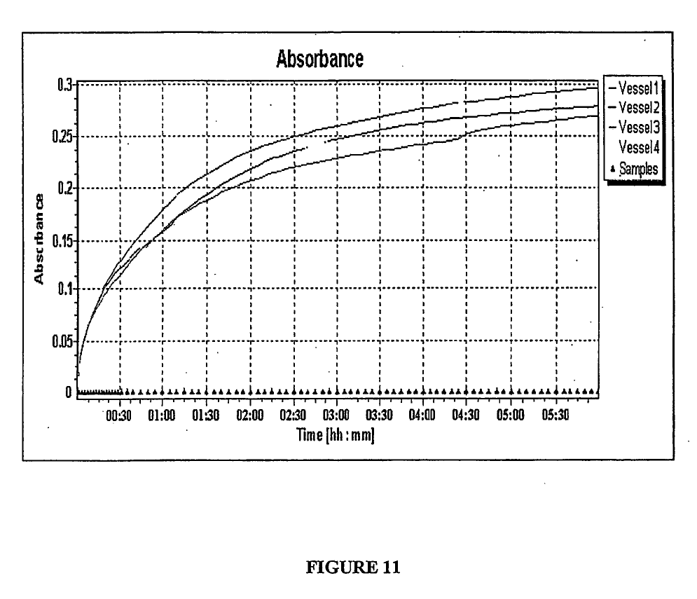

USP type IV dissolution testing was carried out using Sotax CE70 dissolution apparatus, run for six hours at 37° C. and 32 mL min⁻¹. 200 mg samples of the nifedipine coated excipient and 1 L of pH 6.8 phosphate buffer were used in each test. The absorbance of each solution was determined at 238 nm. Comparison with a standard of known concentration indicated that complete dissolution would yield an absorbance value of 0.316. Dissolution of between 85 and 90% (2.25-2.125 mg) was achieved for the four samples tested over the six hour period. The dissolution results for the four samples tested over the six hour period are indicate in FIG. 11.

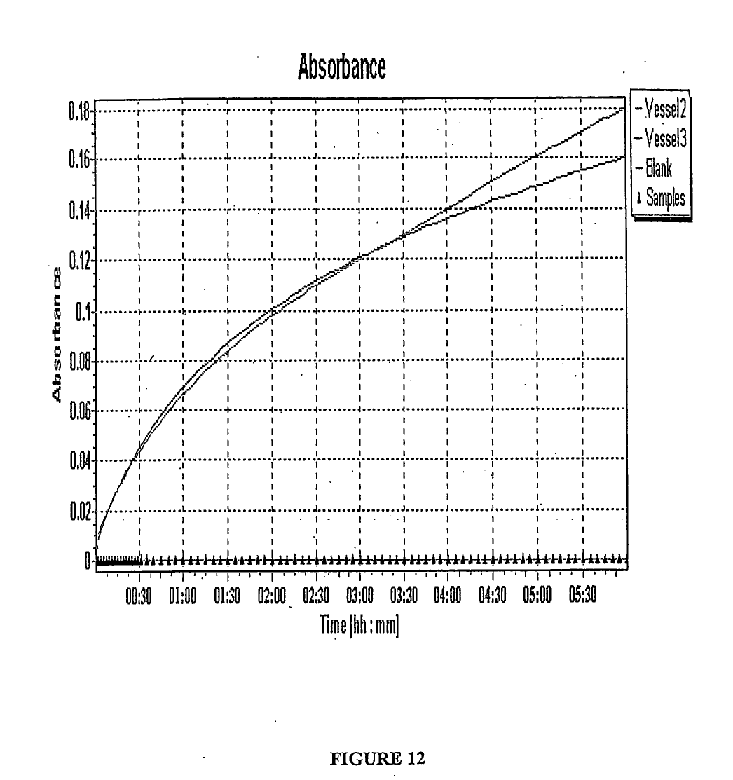

USP grade nifedipine was dissolution tested in order to compare the dissolution results of the nifedipine coated excipient with the dissolution of USP grade nifedipine. Two vessels were used to test the dissolution of the USP grade nifedipine. 10 mg of USP grade nifedipine was used in each test. Conditions were equivalent in both the dissolution test of the coated excipient above and the USP grade nifedipine. The results of the dissolution of the USP grade nifedipine are indicated in FIG. 12. The results of the USP grade nifedipine dissolution test show that after six hours approximately 50% had dissolved (1.25 mg), shown by the absorbance of approximately 0.16-0.18.

Comparing the two dissolution profiles indicates that the rate of dissolution is approximately twice as rapid for the nifedipine coated excipient as for the pure drug (USP grade nifedipine).

### EXAMPLE 13

Preparation of a Calcium Phosphate/Xanthan Gum Matrix Employing Latex Beads as the Pore Forming Agent.

General Methods

Preparation of Reticulated Structures

Between 0.5 and 5% w/v of a pharmaceutically approved gum is dissolved in an aqueous solution of between 15 and 50% w/v sodium phosphate. Between 0.5 and 5% w/v calcium carbonate is then be added to this solution. Latex beads of between 0.1 and 10 μm in diameter are weakly aggregated by allowing a sample to dry until a wet mass can be collected. Between 0.05 and 1% w/v of the weakly aggregated latex beads are then added to the solution. The dispersion is stirred vigorously and between 1 and 20% w/v of an aqueous solution of between 20 and 75% w/v calcium chloride is added to it. The resulting solid is dried at between 15 and 80° C., before being added to a suitable volume of acetone and stirred for between 15 minutes and 24 hours. The sample are then filtered, allowed to dry and broken up roughly to generate a free flowing powder.

Electron Microscopy

A small fraction of the powdered material was carefully placed onto a metal stub and sputter coated with gold in preparation for imaging using a Jeol 6310 scanning electron microscope (SEM). Images were recorded at several magnifications to determine the existence of both primary and secondary level structure.

The specific surface area was measured using a Backman Coulter SA3100 Surface Area and Pore Size Analyzer. Samples of 0.075 to 1.5 g in weight were crushed in a controlled manner, such that the crushed particle size was sufficient that the sample could enter the pre-dried 3 ml glass sample tubes. The samples were dried at 60° C. for 10 hours prior to measurement. Measurement was made using nitrogen gas and the BET equation on 10 pressure points. A minimum of three measurements were made on each sample.

Specific Methods:

(a) 17.2 g of sodium phosphate was dissolved in 50 ml of deionised water. To this solution was added 1.0 g of xanthan gum and 1.0 g of CaCO₃. The solution was stirred until the xanthan gum had dissolved completely in the solution. 0.1 g of solid 300 μm diameter latex beads was then added to the solution. The dispersion was stirred vigorously and an aqueous solution containing 5.28 g of calcium chloride in 10 ml of deionised H₂O was added dropwise. The resulting solid was dried at room temperature and pressure. Following complete dying, the material was added to a suitable volume of acetone, to dissolve the latex beads, and stirred overnight. The sample was then filtered to remove the acetone and dried. The specific surface areas of structures prepared by this method are found, by nitrogen absorption, to be in the range of 0.1-15 m²/g.

(b) 17.2 g of sodium phosphate was dissolved in 50 ml of deionised water. To this solution was added 1.0 g of xanthan gum and 1.0 g of CaCO₃. The solution was stirred until the xanthan gum had dissolved completely in the solution. 2 ml of a 10 wt % suspension of 3 μm diameter latex beads was then added to the solution. The resulting dispersion was stirred vigorously and an aqueous solution containing 5.28 g of calcium chloride in 10 ml of deionised H₂O was added dropwise. The resulting solid was dried at room temperature and pressure. Following complete dying, the material was added to a suitable volume of acetone, to dissolve the latex beads, and stirred overnight. The sample was then filtered to remove the acetone and dried at 95° C. overnight. Specific surface area was determined by nitrogen adsorption using the BET equation and was found to be 21.3 m²/g.

Scanning Electron Microscope Images of Example 13

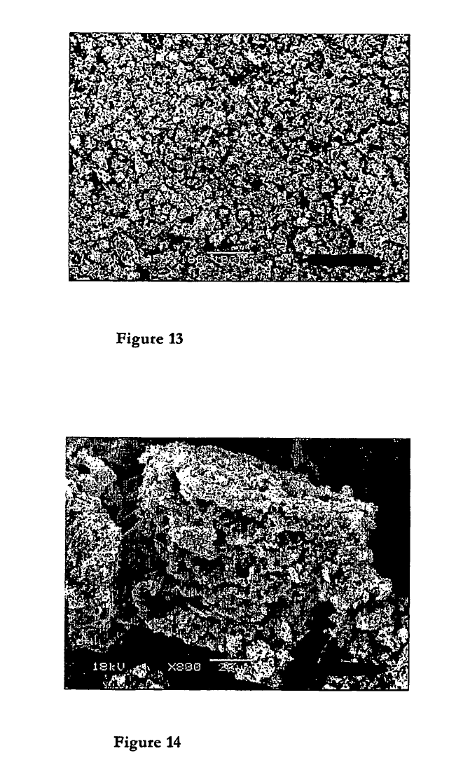

FIG. 13 is taken at a magnification of ×180 and shows the material prepared in example (a). This material comprises clusters of calcium phosphate incorporating spherical pores of 300 nm diameter. The individual clusters abut each other to form a continuous structure defining a primary pore structure in the range of 2 to 50 μm. FIG. 14 is taken at ×800 magnification and shows a single cluster of calcium phosphate prepared using approximately 2 μm latex particles as a porogen in a method in accordance with the general instructions given in this example. The aggregation of individual calcium phosphate, forming the cluster, can clearly be seen.

### EXAMPLE 14

Preparation of Calcium Phosphate and Calcium Carbonate/Egg Albumen Matrices

General Methods

Preparation of Reticulated Structures Using Pre-Formed Particles

Between 5 and 20 g of inorganic particles, either calcium carbonate or calcium phosphate, are added to 10 ml of a solution containing between 5 and 10 % w/w egg albumen. The slurry is then stirred vigorously for 30 minutes to ensure wetting of the particles. The slurry is whisked at high speed using a domestic Kenwood food mixer to incorporate air into the mixture. Whisking is carried out until deformable asperities are observed within the sample and foam is produced. The foam is transferred to an oven and heated at between 40 and 100° C. for between 12 and 24 hours. The material is then cooled to room temperature and broken up roughly to generate a free flowing powder.

Preparation of Reticulated Structures Using in-situ Precipitation

Between 1 and 10 ml of a solution of between 0.1 and 10 mol/dm³ sodium carbonate or sodium phosphate is added to 10 ml of a solution containing between 5 and 10% w/w egg albumen. The solution is then stirred vigorously for up to 10 minutes to ensure equilibrium conditions are achieved. The solution is whisked at high speed using a domestic Kenwood food mixer to incorporate air into the mixture. During whisking, between 1 and 10 ml of a solution of between 0.1 and 10 mol/dm³ calcium chloride is added dropwise. Whisking is carried out until deformable asperities are observed within the sample and foam is produced. The foam is transferred to an oven and heated, at between 40 and 100° C. for between 12 and 24 hours. The material is then cooled to room temperature and broken up roughly to generate a free flowing powder.

Electron Microscopy

A small fraction of the powdered material was carefully placed onto a metal stub and sputter coated with gold in preparation for imaging using a Jeol 6310 scanning electron microscope (SEM). Images were recorded at several magnifications to determine the existence of both primary and secondary level structure.

Specific Surface Area Determination

The specific surface area was measured using a Beckman Coulter SA3100 Surface Area and Pore Size analyser. Samples of 0.075 to 1.5 g in weight were crushed in a controlled manner, such that the crushed particle size was sufficient that the sample would enter the pre-dried 3 ml glass sample tubes. The samples were dried at 60° C. for 10 hours prior to measurement. Measurement was made using nitrogen gas and the BET equation on 10 pressure points. A minimum of three measurements were made on each sample.

Specific Methods

(a) Preparation: 10 g of calcium carbonate particles were added to 10 ml of a solution containing 10% w/w egg albumen. The slurry was then stirred vigorously for 30 minutes to ensure complete wetting of the particles. The slurry was whisked at high speed using a domestic Kenwood food mixer to incorporate air into the mixture. Whisking was carried out until deformable asperities were observed within the sample and foam was produced. The foam was transferred to an over and heated, at 60° C. for 12 hours. The material was then cooled to room temperature and broken up roughly to generate a free flowing powder.

Results: The specific surface area of this material prepared by this method was found, by nitrogen absorption, to be 4.4 m²/g.

(b) Preparation: 5 g of calcium carbonate particles were added to 15 ml of a solution containing 5% w/w egg albumen. The slurry was then stirred vigorously for 30 minutes to ensure complete wetting of the particles. The slurry was whisked at high speed using a domestic Kenwood food mixer to incorporate air into the mixture. Whisking was carried out until deformable asperities were observed within the sample and foam was produced. The foam was transferred to an over and heated, at 40° C. for 24 hours. The material was then cooled to room temperature and broken up roughly to generate a free flowing powder.

Results: The specific surface area of this material prepared by this method was found, by nitrogen absorption, to be 3 m²/g.

Scanning Electron Microscope Images of Example 14

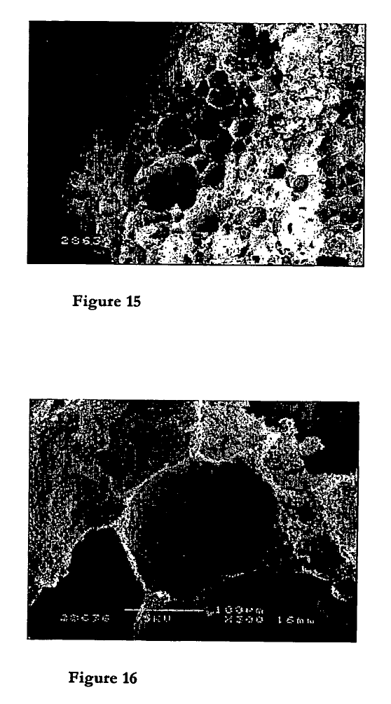

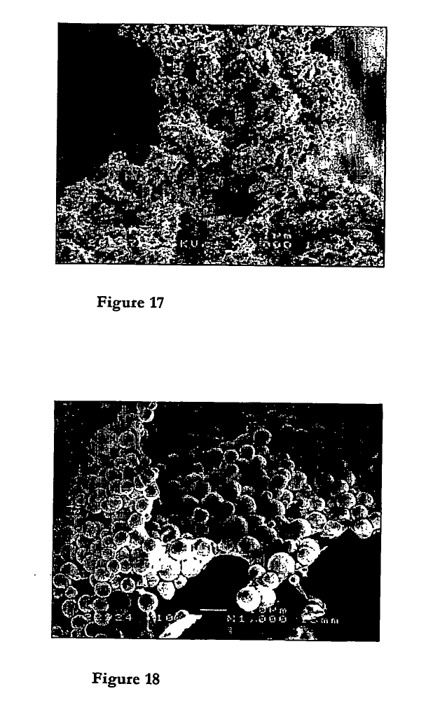

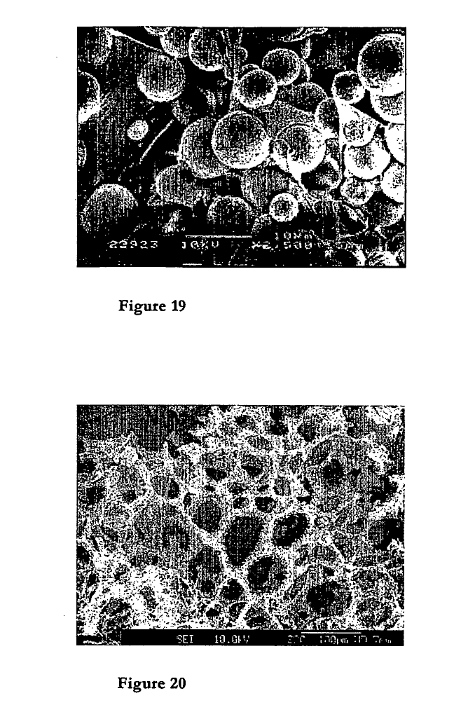

Electron Microscopy. FIG. 15 is taken at ×40 magnification and shows a reticulated structure prepared using pre-formed calcium carbonate particles. It is clear that the calcium carbonate particles are located within the interstitial spaces of the foam and at the interface of each air bubble. A wide “bubble” size distribution can be observed with bubble diameters below approximately 100 μm. A magnified (×300) image of one of the “bubbles”; can be seen in FIG. 16. The particles line up along the surface of the “bubble” forming a highly micro-porous structure with pores in the range of 0.1-5 μm. The walls formed between “bubbles” is shown in FIG. 17 (×5,000 magnification). The wall thickness has been shown to be in the range of 0.5-10 μm, with the walls containing further pores in the range of 0.1-5 μm. A reticulated structure prepared by in situ precipitation is shown in FIG. 18 (at ×1000 magnification). Near-spherical particles of calcium carbonate, in the range of 1-15 μm in diameter can be observed to be orientated along the interface of each air bubble. “Bubble” sizes of the order of 100 μm can be observed. FIG. 19 (×2,500 magnification) shows the loose packing of particles along the interface. Spaces between near-spherical particles act as pores in the wall structure.

Specific Surface Area Determination. The specific surface areas of additional structures prepared by these methods were found, by nitrogen adsorption, to be in the range of 1-15 m²/g.

### EXAMPLE 15

Preparation of Calcium Phosphate Matrices using a High Temperature Process

General Methods

Preparation of Reticulated Structures Using Sub-Micron Hydroxyapatite Particles

Between 0.5 and 5 g of pre-prepared hydroxyapatite particles of sub-micron diameter are suspended in up to 15 ml of distilled water with sonication. To this suspensions is added between 5 and 50 g of dextrin (M, approx 70,000) with mixing. The paste is then air-dried at room temperature and heated to between 900 and 1500° C. for up to 5 hours with a heating rate of between 1 and 20° C./min. Reticulated sponges composed of fused particles of approximately 1 μm in diameter with pore size up to 100 μm are obtained.

Preparation of Reticulated Structures Using 4 Micron Hydroxyapatite

Particles Between 0.5 and 10 g of pre-prepared hydroxyapatite particles of approximately 4 μm in diameter are suspended in up to 50 ml of distilled water with sonication. To this suspension is added between 5 and 50 g of dextrin (M, approx 70,000) with mixing. Epichlorhydrin (1-chloro-2,3-epoxypropane) is stirred into the mixture in the range of 0.5 to 10 ml, to act as a cross-linking agent. Between 0.5 and 5 ml of 5.8 M sodium hydroxide solution is added to the suspension, with mixing, to form a viscous liquid. The liquid is poured into circular moulds and left for between 8 and 24 hours in a covered container. The resulting rubber-like dextran/hydroxyapatite material is then removed from the moulds and placed in a crucible and heated to between 1000 and 1500° C. for up to 5 hours with a heating rate of between 1 and 20° C./min. Sponge like discs of a reticulated framework of hydroxyapatite fused particles are obtained.

Electron Microscopy

A small fraction of the powdered material was carefully placed onto a metal stub and sputter coated with gold in preparation for imaging using a Keol 6310 scanning electron microscope (SEM). Images were recorded at several magnifications to determine the existence of both primary and secondary level structure.

Specific Surface Area Determination

The specific surface area was measured using a Beckman Coulter SA3100 Surface Area and Pore Size Analyser. Samples of 0.075 to 1.5 g in weight were crushed in a controlled manner, such that the crushed particle size was sufficient that the sample would enter the pre-dried 3 ml glass sample tubes. The samples were dried at 60° C. for 10 hours prior to measurement. Measurement was made using nitrogen gas and the BET equation on 10 pressure points. A minimum of three measurements were made on each sample.

Specific Methods

(a) Preparation: 1.5 g of pre-prepared hydroxyapatite particles of sub-micron diameter were suspended 5 ml of distilled water with sonication, 10 g of dextran (M, approx 70,000) was added with mixing. The paste was then air-dried and heated to 1000° C. for 2 hours with a heating rate of 10° C./min. Reticulated sponges composed of fused particles of approximately 1 μm in diameter with pore size up to 100 μm were obtained. In this case heating of the hydroxyapatite particles resulted in a phase change to α-tri-calcium phosphate (α-Ca₃(PO₄)₂).

(b) Preparation: 1.5 g of pre-prepared hydroxyapatite particles of approximately 4 μm in diameter were suspended 4.5 ml of distilled water with sonication, 9 g of dextran (M, approx 70,000) was added with mixing. 1.89 ml of cross-linking agent epichlorhydtin (1-chloro-2,3-epoxypropane) was mixed into the mixture, then 2.9 ml of 5.8M sodium hydroxide solution with mixing to form a viscous liquid. The liquid was poured into 15 mm wide, 4 mm deep circular moulds (approx. 8-10) and left overnight in a covered container. The rubber-like dextran/hydroxyapatite material obtained was then removed from the moulds and then placed in an alumna crucible and heated to 1300° C. for 2 hours with a heating rate of 10° C./min. Sponge like discs of a reticulated framework of hydroxypatite fused particles were obtained.

(c) Preparation: 1.5 g of pre-prepared hydroxyapatite particles of approx 4 μm in diameter were suspended 4.5 ml of distilled water with sonication, 7.5 g of dextran (Mr approx 70,000) was added with mixing. 1.57 ml of cross-linking agent epichlorhydrin (1-chloro-2,3epoxypropane) was mixed into the mixture, then 2.45 ml of 5.8 M sodium hydroxide solution with mixing to form a viscous liquid. The liquid was poured into 15 mm wide, 4 mm deep circular moulds (approx. 8-10) and left overnight in a covered container. The rubber-like dextran/hydroxyapatite material obtained was then removed from the moulds and then placed in an alumina crucible and heated to 1350° C. for 2 hours with a heating rate of 5° C./min. Sponge like discs of a reticulated framework of hydroxyapatite fused particles were obtained.

Scanning Electron Microscope Images of Example 15

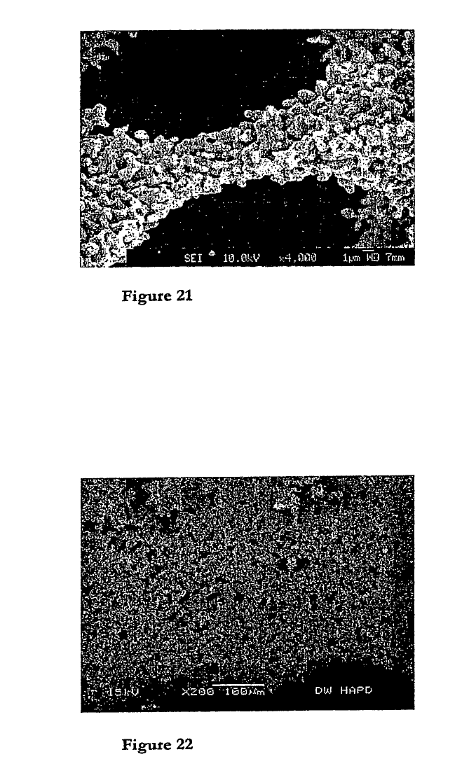

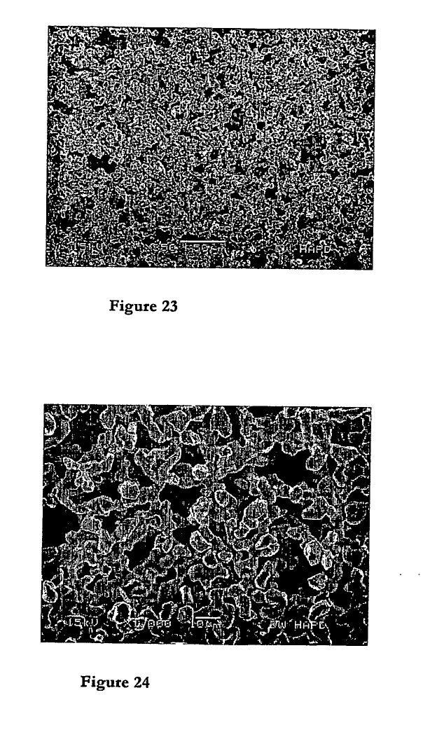

FIG. 20 (×220 magnification) shows a reticulated structure prepared using pre-formed calcium phosphate particles (0.8 to 1 μm) and heating to a temperature of 1,000° C. It is clear that the calcium phosphate particles were located along the strands of the polymeric template, and retain their structure after heating at 1000° C. FIG. 21 (×4,000 magnification) shows a magnified single strand of the structure shown in FIG. 20, consisting of sintered particles and submicron sized pores. There is an absence of particle grain boundaries confirming the successful sintering of the material. FIGS. 22 (×200 magnification) and FIG. 23 (×350 magnification) show the effect of increased temperature (1,350° C.) on the reticulated structure. The mean distance between strands is seen to decrease with increasing temperature. FIG. 24 (×1,000 magnification) shows a magnified portion of the structure seen in FIG. 23. Particle grain boundaries are absent to confirm the effect of sintering.

Specific Surface Area Determination. The specific surface areas of structures prepared by these methods were found, by nitrogen adsorption, to be in the range of 0.1-15 m²/g.

1-146. (canceled)

147. A pharmaceutical excipient comprising a solid, reticulated matrix, wherein the matrix comprises an aggregation of inorganic particles in association with an organic polymeric material, defines a plurality of pores with a mean width in the range of about 0.01-500 μm, and has a specific surface area of at least about 1 m²/g.

148. An excipient as claimed in claim 147, wherein:
(a) the pores have a mean width in the range of about 0.1-500 μm and the matrix has a specific surface area of no more than about 100 m²/g;
(b) the mean width of the pores is in a range of about 0.5-300, 1-200, 3-100, 5-80, 15-70, 20-60 or 0.1-10 μm;
(c) the matrix has a specific surface area of at least about 2, 3, 4, 5, 10 or 20 m²/g, and/or up to about 100, 50 or 40 m²/g;
(d) the inorganic particles are crystalline;
(e) the aggregation of inorganic particles comprises a plurality of discrete crystals; and/or,
(f) the mean width of the inorganic particles is about 0.1-50 μm.

149. An excipient as claimed in claim 147, wherein the pores comprise primary and secondary pores, the primary pores have a mean width of about 2-500 μm and are defined between structural elements formed from the matrix, the secondary pores have a mean width of 0.01-10 μm and are defined within said structural elements, and the mean width of the secondary pores is less than the mean width of the primary pores.

150. A pharmaceutical excipient comprising a solid, reticulated matrix, wherein the matrix comprises an inorganic material in association with an organic polymeric material, a plurality of primary pores with a mean width of about 2-500 μm are defined between structural elements formed from the matrix, a plurality of secondary pores with a mean width of about 0.01-10 μm are defined within said structural elements, and the mean width of the secondary pores is less than the mean width of the primary pores.

151. An excipient as claimed in claim 150, wherein:
(a) the matrix has a specific surface area of at least 0.1, 0.5, 1, 3, 4, 5, 10 m²/g, and/or up to about 100, 50 or 40 m²/g;
(b) the mean width of the primary pores is at least about 5, 10, 20 or 40 μm, and/or no more than about 300, 200, 100 or 50 μm;
(c) the mean width of the secondary pores is at least about 0.01, 0.05, or 0.1 μm, and/or no more than about 5, 3, 2, 1.5 or 1 μm; and/or,
(d) at least about 50, 55, 70, 80, 90, or 95% of the primary pores have a width that is greater than the mean width of the secondary pores, and/or at least about 50, 55, 70, 80, 90, or 95% of the secondary pores have a width that is less than the mean width of the primary pores.

152. An excipient as claimed in claim 149, wherein the structural elements form primary walls that define the primary pores, and comprise a network of secondary walls that define the secondary pores.

153. An excipient as claimed in claim 150, wherein the structural elements form primary walls that define the primary pores, and comprise a network of secondary walls that define the secondary pores.

154. An excipient as claimed in claim 147, wherein the matrix is in the form of a plurality of agglomerations of organic polymeric material and inorganic particles or material, in which the secondary pores are formed, and the primary pores are preferably located between adjacent agglomerations.

155. An excipient as claimed in claim 150, wherein the matrix is in the form of a plurality of agglomerations of organic polymeric material and inorganic particles or material, in which the secondary pores are formed, and the primary pores are preferably located between adjacent agglomerations.

156. An excipient as claimed in claim 149, wherein the inorganic material is particulate, preferably crystalline and, optionally, comprises a plurality of discrete crystals.

157. An excipient as claimed in claim 150, wherein the inorganic material is particulate, preferably crystalline and, optionally, comprises a plurality of discrete crystals.

158. An excipient as claimed in claim 149, wherein the mean width of the organic particles is within the range of about 0.1-50 μm.

159. An excipient as claimed in claim 150, wherein the mean width of the organic particles is within the range of about 0.1-50 μm.

160. An excipient as claimed in claim 147, wherein:
(a) the organic polymeric material binds the inorganic particles or material into the matrix;
(b) the organic polymeric material forms a template for the inorganic particles or material;
(c) the matrix consists essentially or solely of an aggregation of inorganic particles in association with an organic polymeric material;
(d) the organic polymeric material is at least readily soluble in water at a temperature of between 20 and 50° C. within a period of 24 hours;
(e) the polymeric material comprises a polysaccharide and/or a protein; and/or,
(f) the polymeric material comprises xanthan gum, dextran, acacia gum and/or egg albumen.

161. An excipient as claimed in claim 150, wherein:
(a) the organic polymeric material binds the inorganic particles or material into the matrix;
(b) the organic polymeric material forms a template for the inorganic particles or material;
(c) the matrix consists essentially or solely of an aggregation of inorganic particles in association with an organic polymeric material;
(d) the organic polymeric material is at least readily soluble in water at a temperature of between 20 and 50° C. within a period of 24 hours;
(e) the polymeric material comprises a polysaccharide and/or a protein; and/or,
(f) the polymeric material comprises xanthan gum, dextran, acacia gum and/or egg albumen.

162. An excipient as claimed in claim 147 consisting essentially or solely of the matrix, and/or comprising from about 5 to about 95% by weight of said polymeric material or template.

163. An excipient as claimed in claim 150 consisting essentially or solely of the matrix, and/or comprising from about 5 to about 95% by weight of said polymeric material or template.

164. A pharmaceutical excipient comprising a porous network of fused inorganic elements, said network defining a plurality of pores with a mean width within the range of about 0.01-100 μm.

165. An excipient as claimed in claim 18, wherein the inorganic elements have a mean width of no more than about 10, 5 or 2 μm, and/or are at least partially crystalline.

166. An excipient as claimed in claim 164, wherein the pores comprise primary and secondary pores, the primary pores have a mean width of about 2-500 μm and are defined between structural elements formed from the matrix, the secondary pores have a mean width of 0.01-10 μm and are defined within said structural elements, and the mean width of the secondary pores is less than the mean width of the primary pores.

167. An excipient as claimed in claim 166, wherein:
(a) the mean width of the primary pores is at least about 5, 10, 20 or 40 μm, and/or no more than about 300, 200, 100 or 50 μm;
(b) the mean width of the secondary pores is at least about 0.01, 0.05, or 0.1 μm and/or no more than about 5, 3, 2, 1.5 or 1 μm; and/or,
(c) at least about 50, 55, 70, 80, 90, or 95% of the primary pores have a width that is greater than the mean width of the secondary pores and/or at least about 50, 55, 70, 80, 90, or 95% of the secondary pores have a width that is less than the mean width of the primary pores.

168. An excipient as claimed in claim 166, wherein the structural elements form primary walls that define the primary pores, and comprise a network of secondary walls that define the secondary pores.

169. An excipient as claimed in claim 168, wherein the primary walls have a mean width of about 10-500, 10-200, 20-100 or 10-50 μm, and/or the secondary walls have a mean width of about 0.01-5 or 0.5-2 μm.

170. An excipient as claimed in claim 164 comprising a plurality of pores with a mean width of 0.01-50 μm.

171. An excipient as claimed in claim 164 consisting essentially or solely of said fused inorganic elements.

172. An excipient as claimed in claim 147, wherein the inorganic material comprises or the inorganic particles comprise silica and/or a pharmaceutical acceptable alkaline earth metal salt, preferably calcium phosphate and/or calcium carbonate.

173. An excipient as claimed in claim 150, wherein the inorganic material comprises or the inorganic particles comprise silica and/or a pharmaceutical acceptable alkaline earth metal salt, preferably calcium phosphate and/or calcium carbonate.

174. An excipient as claimed in claim 164, wherein the inorganic material comprises or the inorganic particles comprise silica and/or a pharmaceutical acceptable alkaline earth metal salt, preferably calcium phosphate and/or calcium carbonate.

175. A method of preparing a solid, reticulated matrix including the steps of, forming a reticulated template comprising an organic polymeric material, forming a construct comprising an aggregation of inorganic particles in association with said template, and solidifying said construct to form a solid, reticulated matrix comprising the inorganic particles in association with the organic polymeric material, where said matrix defines a plurality of pores with a mean width of 0.01-500 μm and/or has a specific density of at least 1 m²/g.

176. A method as claimed in claim 175, wherein:
(a) the reticulated template, aggregation of inorganic particles and the construct are formed substantially simultaneously; and/or,
(b) the reticulated template is formed by dispersing a second phase in a liquid phase that comprises the organic polymeric material.

177. A method as claimed in claim 176, wherein said second phase comprises or consists essentially of solid particles and/or gas bubbles, and/or the liquid phase comprises a solution of the organic polymeric material.

178. A method as claimed in claim 177, wherein the solid particles are soluble in a solvent in which the organic polymeric material, once set or solidified, is substantially insoluble and the inorganic particles, preferably, are substantially insoluble in said solvent.

179. A method as claimed in claim 175, wherein:
(a) the reticulated template is spontaneously formed from a solution of organic polymeric material, or by the action of a cross-linking agent on a dissolved organic polymeric material;
(b) the reticulated template is formed from an aqueous solution of the polymeric material; and/or,
(c) a proportion of the inorganic particles are formed by precipitation from a solution comprising the organic polymeric material and, preferably, the inorganic particles comprise an inorganic salt and said solution further comprises dissolved anions and/or cations of said salt.

180. A method as claimed in claim 179(c), wherein, before precipitation is initiated, said solution includes dissolved anions but substantially no dissolved cations, or dissolved cations and substantially no dissolved anions of the salt, and precipitation, preferably, is caused by the addition of a solution comprising the counter ions required to form the salt.

181. A method as claimed in claim 179(c), wherein formation of the inorganic particles by precipitation can take place during or after the formation of the reticulated template.

182. A method as claimed in claim 175, wherein at least a proportion of and, optionally, substantially all of the inorganic particles are pre-formed and dispersed in a liquid phase which comprises the organic polymeric material, or substantially all of the inorganic particles are formed by precipitation from a solution comprising the organic polymeric material.

183. A method as claimed in claim 175, wherein:
(a) the inorganic particles are crystalline;
(b) the mean width of the particles or crystals of inorganic material is in the range of about 0.1-50 μm;
(c) the aggregation of inorganic particles forms a part of the reticulated template; and/or,
(d) the construct of reticulated template and aggregation of inorganic particles is solidified by air-drying, spontaneous cross-linking, the action of a cross-linking agent, the effect of a temperature change, the act of forming the inorganic particles by precipitation and/or the influence of electro-magnetic radiation.

184. A method as claimed in claim 175, wherein the reticulated template is formed by entraining gas bubbles in a liquid phase comprising the organic polymeric material, and at least a proportion of the inorganic particles are caused to precipitate during said entrainment process, or the reticulated structure is formed by distributing solid particles in a liquid phase comprising the organic polymeric material and the solid particles are removed after the construct has been solidified by dissolution in an appropriate solvent.

185. A method as claimed in claim 184, wherein the liquid phase comprises a solution of the organic polymeric material.

186. A method as claimed in claim 175, wherein the organic polymeric material includes or is a polysaccharide and/or a protein, and/or the inorganic particles comprise an alkaline earth metal carbonate and/or phosphate and the method, preferably, comprises the steps of forming an aqueous solution of an organic polymeric material and a soluble phosphate or carbonate salt and causing the alkaline earth metal carbonate or phosphate inorganic particles to precipitate from said solution by the addition of an aqueous solution of a soluble salt, preferably a chloride, of the alkaline earth metal.

187. A method as claimed in claim 175, wherein the inorganic particles comprise calcium carbonate and/or calcium phosphate and the organic polymeric material, preferably, is at least readily soluble in water at a temperature between 20 and 50° C. within a period of 24 hours.

188. A method as claimed in claim 175, wherein the polymeric material comprises:
(a) a polysaccharide and/or a protein; and/or,
(b) xanthan gum, dextran, acacia gum and/or egg albumen.

189. A method as claimed in claim 184, wherein the solid particles employed to form the reticulated template are formed from latex.

190. A method for preparing a pharmaceutical excipient, comprising the step of preparing a solid, reticulated matrix by a method as claimed in claim 175.

191. A method for preparing a pharmaceutical excipient as claimed in claim 164, comprising heating a solid, reticulated matrix of organic polymeric material and inorganic particles to a sufficiently high temperature to both eliminate the organic polymeric material and cause the inorganic particles to fuse together into a second solid, reticulated matrix defining a plurality of pores with a mean width of up to 100 μm.

192. A method as claimed in claim 191, wherein:
(a) the organic polymeric material is eliminated and the organic particles caused to fuse by heating the solid, reticulated matrix to a temperature of about 800 to 1600° C.; and/or
(b) the solid, reticulated matrix of organic polymeric material and inorganic particles is prepared, or preparable, by a method as claimed in any of claims 169-183 and/or is a matrix as defined in any of claims 147-157.

193. A method as claimed in claim 191, wherein the solid, reticulated matrix is heated to a temperature of up to about 1100° C. or about 1050° C., or to a temperature in excess of 1100° C., optionally, to a temperature within the range of about 1200 or 1250-1500° C.

194. A pharmaceutical excipient comprising a solid, reticulated matrix prepared or preparable by a method as claimed in claim 175.

195. A pharmaceutical product comprising a pharmaceutical excipient as claimed in claim 147, and a pharmaceutically active agent, optionally wherein the pharmaceutically active agent is particulate and solid.

196. A pharmaceutical product comprising a pharmaceutical excipient as claimed in claim 150, and a pharmaceutically active agent, optionally wherein the pharmaceutically active agent is particulate and solid.

197. A pharmaceutical product comprising a pharmaceutical excipient as claimed in claim 164, and a pharmaceutically active agent wherein, optionally wherein the pharmaceutically active agent is particulate and solid.

198. A pharmaceutical product as claimed in claim 195, wherein:
(a) the pharmaceutically active agent is intimately associated with the excipient;
(b) the pharmaceutically active agent is located within the pores of the solid, reticulated matrix and/or coated onto the matrix or excipient;
(c) the pharmaceutically active agent lies within Class 2 in the FDA adopted Biopharmaceutical Classification System (BCS);
(d) the pharmaceutically active agent has an aqueous solubility of up to about 1 in 30 or 1 in 100 weight/volume, when measured at a temperature in the range of 15 to 25° C.;
(e) the pharmaceutically active agent is crystalline;
(f) the pharmaceutically active agent is particulate, optionally crystalline, and the particles and/or crystals of pharmaceutically active agent have a mean width of about 10 nm-10 μm, 10 nm-5 ∥m or less than about 1 μm;
(g) said product comprises from about 1 to about 50% W/W pharmaceutically active agent;
(h) said product includes an additional pharmaceutically acceptable excipient and/or diluent; and/or,
(i) said product is in the form of an oral solid dosage form, preferably in the form of a powder, capsule or tablet.

199. A pharmaceutical product as claimed in claim 196, wherein:
(a) the pharmaceutically active agent is intimately associated with the excipient;
(b) the pharmaceutically active agent is located within the pores of the solid, reticulated matrix and/or coated onto the matrix or excipient;
(c) the pharmaceutically active agent lies within Class 2 in the FDA adopted Biopharmaceutical Classification System (BCS);
(d) the pharmaceutically active agent has an aqueous solubility of up to about 1 in 30 or 1 in 100 weight/volume, when measured at a temperature in the range of 15 to 25° C.;
(e) the pharmaceutically active agent is crystalline;
(f) the pharmaceutically active agent is particulate, optionally crystalline, and the particles and/or crystals of pharmaceutically active agent have a mean width of about 10 nm-10 μm, 10 nm-5 μm or less than about 1 μm;
(g) said product comprises from about 1 to about 50% W/W pharmaceutically active agent;
(h) said product includes an additional pharmaceutically acceptable excipient and/or diluent; and/or,
(i) said product is in the form of an oral solid dosage form, preferably in the form of a powder, capsule or tablet.

200. A pharmaceutical product as claimed in claim 197, wherein:
(a) the pharmaceutically active agent is intimately associated with the excipient;
(b) the pharmaceutically active agent is located within the pores of the solid, reticulated matrix and/or coated onto the matrix or excipient;
(c) the pharmaceutically active agent lies within Class 2 in the FDA adopted Biopharmaceutical Classification System (BCS);
(d) the pharmaceutically active agent has an aqueous solubility of up to about 1 in 30 or 1 in 100 weight/volume, when measured at a temperature in the range of 15 to 25° C.;
(e) the pharmaceutically active agent is crystalline;
(f) the pharmaceutically active agent is particulate, optionally crystalline, and the particles and/or crystals of pharmaceutically active agent have a mean width of about 10 nm-10 μm, 10 nm-5 μm or less than about 1 μm;
(g) said product comprises from about 1 to about 50% W/W pharmaceutically active agent;
(h) said product includes an additional pharmaceutically acceptable excipient and/or diluent; and/or,
(i) said product is in the form of an oral solid dosage form, preferably in the form of a powder, capsule or tablet.
